(19) United States
(12) Patent Application Publication
(10) Pub. No.: US 2005/0191283 A1
(43) Pub. Date: Sep. 1, 2005

(54) Methods of treating NFAT-related disorders

(76) Inventors: Suzanne Kadereit, Brookline, MA (US); Mary J. Laughlin, Shaker Heights, OH (US)

Correspondence Address: FISH & NEAVE IP GROUP;ROPES & GRAY LLP, ONE INTERNATIONAL PLACE, BOSTON, MA, 02110-2624, US

(21) Appl. No.: 10/967,649

(22) Filed: Oct. 18, 2004

Related U.S. Application Data
(60) Provisional application No. 60/512,248, filed on Oct. 16, 2003.

(51) Int. Cl. A61K 48/00

(52) U.S. Cl. 424/93.21; 514/44

(57) ABSTRACT

The invention provides, among other things, methods for modulating NFATc2 and methods for treating graft versus host disease, transplant rejection, and other NFATc2-related disorders.

### CROSS-REFERENCE TO RELATED APPLICATIONS

This application claims the benefit of the filing date of U.S. Provisional Application No. 60/512248, filed Oct. 16, 2003, entitled “METHODS OF TREATING NFAT-RELATED DISORDERS.” The entire teachings of the referenced application are incorporated by reference herein.

### FUNDING

The invention described herein was supported, in whole or in part, by grant RO1-AI47289-01 from the National Institutes of Health. The United States government has certain rights in the invention.

### BACKGROUND OF THE INVENTION

The NFAT (nuclear factor of activated T cells) family of transcription factors participate in a wide range of biological processes. One member of the family, NFATc2 (also termed NFAT-1) is a key regulator of the immune system and is also involved in the growth and differentiation of a variety of cell types, particularly the myocytes of skeletal and heart muscle. The NFATc2 transcription factor resides in latent form in the cytoplasm of T cells and other cell types. Upon T-cell activation, NFATc2 proteins are dephosphorylated by calcineurin, a Ca²⁺-dependent phosphatase, and translocate into the nucleus to activate the target genes (Rao et al. Ann. Rev. Immunol. 15:707-747, 1997). While regulation of NFATc2 protein activation and its role in transcriptional activation of numerous genes has been studied extensively (Crabtree et al. Cell 109:S67-79, 2002), little is known about the regulation of the NFATc2 gene itself. In T cells, NFATc2 mRNA expression is not significantly upregulated immediately (2-3 h) after stimulation (Lyakh et al. Mol Cell Biol 17:2475-2484, 1997; Northrop et al. Nature 369:497-502, 1994). However, NFATc2 mRNA and protein levels increase during prolonged stimulation.

Therefore, there is a pressing need in the art to identify agents that modulate NFATc2 and thereby modulate NFATc2-regulated processes. Such agents may be used, for example, to treat disorders of the immune system, as well as other disorders associated with NFATc2 activity.

### SUMMARY OF THE INVENTION

In certain aspects, the present invention provides novel methods of regulating immune responses and other NFAT-related processes. Methods of the present invention are based, in part, on the discovery that interferon gamma (IFN-γ) regulates the expression level and activity of the transcription factor NFATc2. Methods for increasing the activity of NFATc2 in a lymphocyte through the use of IFN-γ agonists, and methods for decreasing the activity of NFATc2 in a lymphocyte through the use of IFN-γ antagonists are disclosed. Methods using an IFN-γ antagonists for the treatment or prevention of an immunological incompatibility, such as graft versus host disease (GVHD) or organ transplant rejections, are described herein.

One aspect of the invention provides a method of decreasing NFATc2 activity in a cell, particularly a human cell, comprising exposing the cell to an antagonist of IFN-γ, thereby decreasing the activity of NFATc2 in the cell. An IFN-γ antagonist may be essentially any substance that directly or indirectly decreases IFN-γ signaling. In certain embodiments, an IFN-γ antagonist directly or indirectly decreases IFN-γ-mediated stimulation of NFATc2. An IFN-γ antagonist may bind to IFN-γ. Examples of such antagonists include anti-IFN-γ antibodies, such as humanized or fully human anti-IFN-γ antibodies, and small molecules that bind to IFN-γ. An IFN-γ antagonist may be a competitive or non-competitive inhibitor of IFN-γ binding to an IFN-γ receptor polypeptide. An IFN-γ antagonist may inhibit intracellular signaling mediated by an IFN-γ receptor. For example, an IFN-γ antagonist may interfere with JAK-STAT pathway signaling events. An IFN-γ antagonist may decrease the amount of IFN-γ. For example, an antagonist may decrease IFN-γ production (e.g., an antisense or siRNA targeted to IFN-γ gene or transcript). As another example, an antagonist may increase IFN-γ degradation or otherwise decrease IFN-γ bioavailability or serum half-life. An IFN-γ antagonist need not act directly on the cell in which NFATc2 activity is decreased; an IFN-γ antagonist may affect a secondary cell, which in turn affects the target cell. For example, an IFN-γ antagonist may affect an Antigen Presenting Cell (APC) which in turn affects a lymphocyte, such as a T cell. Accordingly, a cell may be considered exposed to an IFN-γ antagonist as long as the cell is affected, directly or indirectly, by the IFN-γ antagonist. In certain preferred embodiments, a cell and one or more secondary cells are, together, exposed to the IFN-γ antagonist. For example, the cell may be present in a mixed cell culture, in a cell mixture comprising hematopoietic stem cells for transplant, in an ex vivo organ, or in an organism.

One aspect of the invention provides a method of increasing NFATc2 activity in a cell, particularly a human cell, comprising exposing the lymphocyte to an agonist of IFN-γ, thereby increasing the activity of NFATc2 in the cell. An IFN-γ agonist may be essentially any substance that directly or indirectly increases IFN-γ signaling. In certain embodiments, an IFN-γ agonist directly or indirectly increases IFN-γ-mediated stimulation of NFATc2. An IFN-γ agonist may comprise an IFN-γ polypeptide (or dimer or other multimer thereof). For example, an IFN-γ agonist may be similar to or identical to naturally occurring IFN-γ, whether purified from a natural source or produced recombinantly (e.g. a 34 kDa homodimer). An IFN-γ agonist may comprise a modified IFN-γ polypeptide, such as a mutant, allelic variant, or pegylated or glycosylated form that retains IFN-γ signaling activity. An IFN-γ agonist may comprise a modified IFN-γ polypeptide, such as a pegylated or glycosylated form that retains IFN-γ signaling activity. An IFN-γ agonist may comprise a functionally active portion of an IFN-γ polypeptide, which may also include one or more modifications. An IFN-γ agonist may be a substance (e.g., peptidomimetic, small molecule) that binds to and activates an IFN-γ receptor. An IFN-γ agonist may activate intracellular signaling mediated by an IFN-γ receptor. For example, an IFN-γ agonist may stimulate JAK-STAT pathway signaling events. An IFN-γ agonist may increase the amount of IFN-γ. For example, an agonist may increase IFN-γ production (e.g., a highly expressed transgene encoding IFN-γ). As another example, an agonist may decrease IFN-γ degradation or otherwise increase IFN-γ bioavailability or serum half-life. An IFN-γ agonist need not act directly on the cell in which NFATc2 activity is increased; an IFN-γ agonist may affect a secondary cell, which in turn affects the target cell. For example, an IFN-γ agonist may affect an Antigen Presenting Cell (APC) which in turn affects a lymphocyte, such as a T cell. Accordingly, a cell may be considered exposed to an IFN-γ agonist as long as the cell is affected, directly or indirectly, by the IFN-γ agonist. In certain preferred embodiments, a cell and one or more secondary cells are, together, exposed to the IFN-γ agonist. For example, the cell may be present in a mixed cell culture, in a cell mixture comprising hematopoietic stem cells for transplant, in an ex vivo organ, or in an organism.

In certain embodiments, an NFATc2 activity to be decreased or increased is an activity that has a clinically or scientifically meaningful effect on a biological process. NFATc2 is known to participate in a wide range of biological processes, including immune responses, angiogenesis, skeletal muscle development and cartilage development. At the molecular level, NFATc2 activity may be assessed by detecting expression of one or more NFATc2 regulated genes (or proteins encoded therein), such as IL-3, IL-4, IL-5, IL-13, TNF, CD40L, GM-CSF, MIP-1α, CCNA2, CCNE2 and p21. At the cellular level, NFATc2 activity may be assessed by analyzing cellular differentiation (e.g., differentiation of mesenchymal stem cell into a chondrocyte, or the activation of a T cell) or global gene or protein expression patterns. Methods disclosed herein are not limited to any particular molecular mechanism(s) by which NFATc2 activity is modulated. NFATc2 activity may, for example, be affected at the transcriptional level, at the level of protein localization, protein phosphorylation, interaction with partners in the formation of transcriptional regulatory complexes, etc. A modulator of NFATc2 activity may affect NFATc2 directly and/or indirectly, and by a plurality of molecular mechanisms.

IFN-γ agonists and antagonists may be used to modulate NFATc2 in essentially any responsive cell type. A cell may be directly or indirectly responsive to the IFN-γ agonist or antagonist. In a preferred embodiments, IFN-γ agonists and antagonists are used to modulate NFATc2 in lymphocytes, such as T cells. In a particularly preferred embodiment, the cell is a transplanted cell, or a cell that is intended for use as a transplant, or a cell that is derived from a transplanted cell.

In certain aspects, the invention provides a method of preventing or reducing immunological incompatibility in a subject in need thereof. A method may comprise administering to the subject a therapeutically effective amount of an IFN-γ antagonist. In a preferred embodiment, the subject has or is at risk for having graft versus host disease (GVHD). A subject may be considered at risk for GVHD if the subject has received a bone marrow transplant or other transplant comprising T cells or precursors thereof (e.g., a transplant comprising hematopoietic stem cells). In another preferred embodiment, the subject has or is at risk for having graft rejection. In certain embodiments, the invention provides methods for preventing or reducing immunological incompatibility in a transplant recipient by administering to the subject a therapeutically effective amount of an IFN-γ antagonist. The transplant recipient may have received a bone marrow transplant or other transplant comprising T cells or precursor cells thereof. The transplant recipient may have received a solid organ transplant. The transplant may be HLA-matched or HLA-unmatched, or allogeneic. In certain embodiments, the transplant is a transplant comprising hematopoietic stem cells. In certain embodiments the transplant is a lung, heart, kidney, liver, skin, or bone marrow transplant. The transplant recipient may have received multiple organs (e.g., heart-lung transplant).

Yet another aspect of the invention provides a method of preventing graft versus host disease in a subject in need of such treatment, the method comprising contacting the transplant, prior to transplantation into the subject, with an IFN-γ antagonist, thereby preventing graft versus host disease in the subject. The transplant may be HLA-matched or HLA-unmatched, or allogeneic. The transplant may be a solid organ comprising T cells or precursors thereof, such as lung, heart, kidney, liver, skin, or bone marrow. The transplant may comprise hematopoietic stem cells, such as hematopoetic stem cells from an unrelated donor, umbilical vein hematopoetic stem cells, or peripheral blood stem cells.

In certain aspects, the invention further provides a method of treating an autoimmune disease in a subject in need thereof, the method comprising administering to the subject a therapeutically effective amount of an IFN-γ antagonist.

In further aspects, the invention provides methods for assessing a candidate activator of NFATc2. A method may comprise providing a candidate agent that is an IFN-γ agonist; and measuring an effect of the candidate agent on an NFATc2 activity. Generally, a candidate agent that causes in increase in NFATc2 activity will be considered an activator of NFATc2. NFATc2 activity may be assessed in cell culture or in an animal or other assay system. In a preferred embodiment, measuring an effect of the agent on an NFATc2 activity comprises measuring expression of NFATc2 or an NFATc2-regulated gene in an umbilical cord blood T cell culture. Measuring NFATc2 activity may also include measuring a proxy for NFATc2 activity (e.g., a phenomenon that is highly correlated with NFATc2 activity) such as a shift in localization from the cytoplasm to the nucleus, or a shift in phosphorylation state. A candidate agent may be a known IFN-γ agonist, or providing an IFN-γ agonist may comprise screening a plurality of agents to identify an agent having IFN-γ agonist activity. A method may comprise further assessments of the activator, such as evaluations of effects on in vivo or in vitro disease models. A preferred disease model is a model for graft versus host disease. An additional preferred disease model is a model for graft rejection. A method may further comprise evaluating the effect of the candidate agent on graft versus host disease or transplant rejection in an animal. A method may further comprise regulated clinical trials in volunteer human subjects.

In further aspects, the invention provides methods for assessing a candidate inhibitor of NFATc2. A method may comprise providing a candidate agent that is an IFN-γ antagonist; and measuring an effect of the candidate agent on an NFATc2 activity. Generally, a candidate agent that causes a decrease in NFATc2 activity will be considered an inhibitor of NFATc2. NFATc2 activity may be assessed in cell culture or in an animal or other assay system. In a preferred embodiment, measuring an effect of the agent on an NFATc2 activity comprises measuring expression of NFATc2 or an NFATc2-regulated gene in an umbilical cord blood T cell culture. Measuring NFATc2 activity may also include measuring a proxy for NFATc2 activity (e.g., a phenomenon that is highly correlated with NFATc2 activity) such as a shift in localization from the cytoplasm to the nucleus, or a shift in phosphorylation state. A candidate agent may be a known IFN-γ antagonist, or providing an IFN-γ antagonist may comprise screening a plurality of agents to identify an agent having IFN-γ antagonist activity. A method may comprise further assessments of the inhibitor, such as evaluations of effects on in vivo or in vitro disease models. A preferred disease model is a model for graft versus host disease. An additional preferred disease model is a model for graft rejection. A method may further comprise evaluating the effect of the candidate agent on graft versus host disease or transplant rejection in an animal. A method may further comprise regulated clinical trials in volunteer human subjects.

In certain aspects, the invention also provides methods for increasing or decreasing the production of NFATc2-dependent cytokines in a subject. One aspect of the invention provides a method of decreasing production of an NFATc2-dependent cytokine in a subject in need of such treatment, comprising administering to the subject a therapeutically effective amount of an IFN-γ antagonist, thereby decreasing the production of the NFATc2-dependent cytokine. Another aspect of the invention provides a method of increasing production of an NFATc2-dependent cytokine in a T cell in a subject in need of such treatment, comprising administering to the subject a therapeutically effective amount of an IFN-γ agonist polypeptide in the subject, thereby increasing the production of the NFATc2-dependent cytokine in the T cell.

In certain aspects, the invention provides a method of preventing or reducing immune incompatibility in a subject in need thereof, the method comprising administering to the subject a therapeutically effective amount of an inhibitor RNA construct that decreases expression of NFATc2 or an NFATc2-regulated factor. An inhibitor RNA construct may also be referred to as an siRNA construct, including short double stranded RNAs and hairpin RNAs with a short region of internal complementarity. In a preferred embodiment, the NFATc2-regulated factor is a factor that is regulated by NFATc2 in cells of umbilical cord blood. Examples of such factors include: GM-CSF, CTLA-4, IFN-γ, IL-2Rα, IL-3, IL-4, IL-5, IL-13, TNF, CD40L, MIP-1α, p21, CCNA2 and CCNE2.

The invention also provides an use for an IFN-γ agonist or antagonist for the manufacture of a medicament to treat immune incompatibilities in a subject. In one embodiment, an IFN-γ antagonist, such as an antibody that binds IFN-γ, an antibody that binds to the IFN-γ receptor, an inhibitory RNA, or a small molecule, is used in the manufacture of a medicament to treat GVHD or organ transplant rejection. The invention further provides an use in manufacturing a medicament for all the methods described herein.

### BRIEF DESCRIPTION OF THE DRAWINGS

FIGS. 1A and 1B show a reduced upregulation of NFATc2 protein in UCB T lymphocytes during prolonged stimulation. MNC from fresh cord and adult peripheral blood were stimulated with ConA for the indicated time points and T cells purified. Whole cell extracts were analyzed for NFATc2 protein expression by immunoblotting with anti-NFATc2 and β-actin mAbs. A) Gel loading was normalized with β-actin, and NFATc2 band intensity expressed in densitometer read-out units. Representative of 11 experiments, comparing each 1 cord blood unit and 1 adult. B) Graphic representation of the 11 experiments from A). For graphing purposes, after normalization for gel loading, NFATc2 protein band intensities were expressed as fold increase over basal (0 h) expression in the control adult, determined as 1. Fold-increase values were plotted in a smoothed scatterplot (Lowess Smoother) (Cleveland W S. J Amer Statist Assoc 74:829-836, 1979.

FIGS. 2A-2C show that NFATc2 upregulation in adult T lymphocytes is IFN-γ dependent. A) Adult MNC were stimulated for 24 h in presence or absence of neutralizing anti-IFN-γ antibodies (1 μg/ml) and NFATc2 protein expression analyzed by intracellular flow cytometry with FITC-conjugated NFATc2 monoclonal antibody, in gated CD3⁺ cells. Numbers indicate percentages of CD3⁺ cells positive for NFATc2-FITC fluorescence. Representative of 6 adults analyzed, demonstrating similar inhibition, by anti-IFN-γ antibody concentrations ranging from 0.25 μg/ml to 2 μg/ml. B) Adult MNC were stimulated as above in presence or absence of CsA to inhibit IFN-γ production during stimulation, and NFATc2 expression analyzed as above. Representative of 3 adults analyzed. C) Adult MNC were stimulated as above for up to 96 h in presence or absence of CsA, T cells purified, and NFATc2 protein expression analyzed by immunoblotting with anti-NFATc2 and β-actin mAbs. Representative of 11 adults analyzed.

FIGS. 3A-3B show that addition of exogenous IFN-γ during stimulation increases NFATc2 expression in UCB and adult T lymphocytes. A) MNC from UCB were stimulated for 16 h with ConA in presence of increasing doses of exogenous IFN-γ(10-1,000 U/ml) and NFATc2 expression analyzed in gated CD3⁺ cells. Representative of 3 similar experiments. B) MNC from adult were stimulated for 24 h in absence or presence of IFN-γ (100 U/ml) and NFATc2 expression analyzed in gated CD3⁺ cells.

Representative of 5 Adults Analyzed.

FIGS. 4A-4B show rescue of IFN-γ production in UCB T lymphocytes during primary stimulation by IFN-γ but not by TNF-A. A) MNC from cord and adult were stimulated with ConA for 24 h with added IFN-γ(100 U/ml) or TNF-α (1 ng/ml) and intracellular IFN-γ expression was analyzed in gated CD3⁺ cells. Numbers represent percentages of CD3⁺/CD69⁺ cells co-expressing IFN-γ. Representative of 20 cord bloods analyzed. B) MNC from cord blood were incubated in RPMI (unstim.), with 100 U/ml of IFN-γ (IFN-γ), with ConA (ConA) or with ConA and 100 U/ml of IFN-γ (ConA+IFN-γ), analyzed by dual intracellular staining for NFATc2 and IFN-γ expression in gated CD3⁺ cells and data expressed as NFATc2-positivity versus IFN-γ-positivity. Numbers indicate the percentage of CD3⁺-gated events within the respective quadrant. Representative of 6 similar experiments.

FIGS. 5A-5C show that IFN-γ-induced expression of IFN-γ in adult and UCB T cells is dependent on accessory cells. A) Alloantigen-specific IFN-γ secretion by UCB T cells was analyzed by Elispot assays in response to 24 h of stimulation by adult or UCB T-cell-depleted and irradiated MNC. Are shown numbers of IFN-γ secreting spots by two different UCB units in response to allostimulation with T-cell-depleted MNC from adult or with MNC from one of the two UCB units (UCB#2). B) Purified (>98%) T cells from adult were stimulated for 24 h with anti-CD3/CD28 antibodies, in presence or absence of IFN-γ(100 U/ml), and intracellular IFN-γ expression measured in gated CD3⁺ cells. Numbers in histograms indicate percentages of CD3⁺ cells expressing IFN-γ. C) MNC from UCB were stimulated for 24 h either in bulk with ConA, in presence or absence of IFN-γ, or T cells were purified (>95%) from the same MNC preparations and stimulated with anti-CD3/CD28 antibodies, in presence or absence of IFN-γ(100 U/ml).

FIG. 6 shows IFN-γ -induced expression of IFN-γ in UCB T cells is inhibited by blocking of the secretory pathway. MNC from UCB were stimulated with ConA in presence or absence of IFN-γ (100 U/ml). Brefeldin A was either added for the last 6 h of stimulation (BFA 6 h), or immediately (BFA 24 h).

FIG. 7 shows NFAT-dependent genes with differential expression in UCB vs. AB CD4+ T-cells

FIG. 8 shows NFAT-associated pathway genes with differential expression in UCB vs. AB CD4+ T-cells

FIG. 9 shows differential expression of cytokine and cytokine receptor genes in UCB vs. AB CD4+ T-cells.

FIG. 10 shows differential expression of chemokine and chemokine receptor genes in UCB vs. AB CD4+ T-cells

FIG. 11 shows the cytokine expression profile of UCB vs. AB stimulated T-cell supernatants.

FIG. 12 shows the analysis of changes in gene expression observed in unstimulated and stimulated CD4+ T-cells isolated from UCB and AB. The changes in gene expression were assessed using the HG-U133A&B microarray. One-way hierarchical clustering (A) was performed on the trimmed data set using GeneSpring v5.1 following per gene normalizations of the Signal. Each gene or probe set was normalized to the median of its Signal across all arrays. The gene tree was constructed using the 2521 probe sets which met the inclusion criteria* in at least one of the three time points. Red and green indicate increased and decreased expression relative to the median, respectively. The Pearson correlation with a minimum distance of 0.001 was used in constructing the gene tree. (B) A Benn Diagram depicts the total number of probe sets meeting inclusion criteria* at each time point.
*At least one P call and FC≧2 and Change≠NC.

FIG. 13 shows a corroboration of differential mRNA expression of selected probes by multiprobe RNase protection analysis. The same total RNA pools used for the gene array studies were analyzed for mRNA expression by RNase protection arrays (A). 5 ug of each pool of RNA was used per hybridization to hCK1, hCK2b and hStress multiprobe templates. Exposure times varied among the gels depending upon the strength of each signal. L32 mRNA is included as a control for input RNA. Representative results from 2-3 repeat experiments are shown. (B) Microarray expression data from the HG-U133A&B array corresponding to the above genes were graphed individually, showing the raw Signal.

FIG. 14 shows decreased surface expression of CXCR4 and CD40L in stimulated UCB CD4+ T-cells. CD4+ T-cells were purified from each one UCB and one AB, stimulated for 20 hours and analyzed by flow cytometry for surface expression of (A) CXCR4 and (B) CD40L. Representative histograms of 4 UCB and 4 adults are shown.

FIG. 15 shows impaired basal expression of transcription factors associated with Th1 and Th2 phenotype in unstimulated UCB. Th1 (STAT4 and t-bet) and Th2 (c-maf) associated transcription factors are decreased in expression in UCB at 0 hr. All three genes met the criteria for inclusion as described in Materials and Methods. Data is shown here as the raw Signal obtained from the Affymetrix HGU133A&B microarray. Fold changes between UCB and AB are indicated (FC).

### DETAILED DESCRIPTION OF THE INVENTION

1. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “antibody” as used herein is intended to include whole antibodies, e.g., of any isotype (IgG, IgA, IgM, IgE, etc), and includes fragments thereof which are also specifically reactive with a target protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies. Antibodies may be humanized and the term is also intended to encompass engineered complexes that comprise antibody-derived binding sites, such as diabodies and triabodies.

The term “including” is used herein to mean, and is used interchangeably with, the phrase “including but not limited” to.

As used herein, the term “nucleic acid” refers to polynucleotides such as deoxyribonucleic acid (DNA), and, where appropriate, ribonucleic acid (RNA). The term should also be understood to include analogs of either RNA or DNA made from nucleotide analogs, and, as applicable to the embodiment being described, single-stranded (such as sense or antisense) and double-stranded polynucleotides.

The term “or” is used herein to mean, and is used interchangeably with, the term “and/or,” unless context clearly indicates otherwise.

The term “such as” is used herein to mean, and is used interchangeably, with the phrase “such as but not limited to”.

The terms “polypeptide” and “protein” are used interchangeably herein.

2. Methods of Regulating NFATc2

The invention provides, in part, novel methods of modulating NFATc2 activity in a lymphocyte. One aspect of the invention provides a method of decreasing NFATc2 activity in a lymphocyte, comprising exposing the lymphocyte to an antagonist of IFN-γ, thereby decreasing the activity of NFATc2 in the T cell. A related aspect of the invention provides a method of increasing NFATc2 activity in a lymphocyte, comprising exposing the lymphocyte to an agonist of IFN-γ, thereby increasing the activity of NFATc2 in the T cell.

In one embodiment of these methods, the lymphocyte is a T cell. In another embodiment, decreasing NFATc2 activity comprises decreasing NFATc2 expression, while increasing NFATc2 activity comprises increasing NFATc2 expression. In a preferred embodiment, the T cell is exposed to an antagonist of IFN-γ in the presence of antigen presenting cells. Antigen presenting cells include, but are not limited to, monocytes, B-cells, macrophages and natural killer (NK) cells. In a preferred embodiment, the T cell of the methods described herein is in a subject.

Exposing a lymphocyte to an antagonist/antagonist comprises in some embodiments adding the antagonist/agonist to the medium in which the cell is found, such as cell culture medium when the exposure is done ex vivo, such that a physical interaction is enabled. In other embodiment, such as when the a agonist/antagonist exposure is effected in vivo in a patient, the agonist/antagonist may be more generally administered to the patient. In another embodiment, the lymphocyte may be exposed in vitro to an antagonist/antagonist, and then introduced into the patient. The agonist/antagonist may then be further administered to the patient, such that the lymphocyte is exposed to the agonist/antagonist both in vitro and in a patient.

In one embodiment of the methods described herein to modulate the activity of NFATc2, the lymphocyte is not endogenous to the subject. In a further embodiment, the lymphocyte or a progenitor thereof may have been transplanted from another subject, or may have been cultured in vitro. For example, a lymphocyte may be generated upon differentiation of a stem cell in vitro, treated with the agonist/antagonist, and then be transplanted into the patient. The stem cell giving rise to the lymphocyte may be derived from the subject or from another subject. In some embodiments, the lymphocyte is contained in transplantable material, such a body organs or bone marrow. Body organs include lung, heart, kidney, liver, pancreas and skin.

The invention further provides methods of treating or preventing immune incompatibilities. One aspect of the invention provides method of preventing or reducing immune incompatibility in a subject in need thereof, the method comprising administering to the subject a therapeutically effective amount of an IFN-γ antagonist. Likewise the invention provides a method of preventing immune incompatibilities in a subject in need of such treatment, the method comprising contacting a transplant, prior to transplantation into the subject, with an IFN-γ antagonist, thereby preventing an immune incompatibility in the subject.

Immune incompatibility comprises cases where cells, preferably human cells from another subject, are introduced into a subject which are not compatible with the subject's immune system and thus are attacked and rejected by the subject's immune system, such as in organ rejection. Immune incompatibilities also comprise cases where immune system cells are introduced into a subject, preferably from another subject, which are not compatible with the subject's cells and thus the introduced immune cells attack the subject's cells, such as in graft versus host disease.

Thus, in one embodiment, the subject has or is at risk for having graft versus host disease (GVHD). GVHD may be acute or it may be chronic. In another embodiment, the subject has or is at risk for graft rejection. In other embodiments, the subject is a recipient of a transplant, such as a solid organ transplant. Solid organ transplants include lung, heart, kidney, liver and skin transplants. Alternatively, the transplant may be a hematopoietic stem cell transplant, such as one from an unrelated donor. The transplant may also comprise umbilical vein hematopoetic stem cells, or peripheral blood stem cells.

The transplants described herein can be HLA-matched or HLA-unmatched. In some embodiments, the transplants described herein are allogenic transplant.

The invention further provides a method of decreasing production of an NFATc2-dependent cytokine in a subject in need of such treatment, comprising administering to the subject a therapeutically effective amount of an IFN-γ antagonist, thereby decreasing the production of the NFATc2-dependent cytokine. Likewise, the invention provides a method of increasing production of an NFATc2-dependent cytokine in a T cell in a subject in need of such treatment, comprising administering to the subject a therapeutically effective amount of an IFN-γ agonist polypeptide in the subject, thereby increasing the production of the NFATc2-dependent cytokine in the T cell. In one embodiment, the NFATc2-dependent cytokine is IFN-γ, TNF-α and IL-2. In another embodiment, the NFATc2-dependent cytokine is not IFN-γ.

In a further embodiment of the method for decreasing production of an NFATc2-dependent cytokine in a subject in need of such treatment, the subject is afflicted with an autoimmune disease. Autoimmune diseases include, but are not limited to primary myxoedema, thyrotoxicosis, pernicious anaemia, autoimmune atrophic gastris, Addison's disease, IDDM, Goodpasture's syndrome, myasthenia gravis, sympathetic ophthalmia, MS, autoimmune haemolytic anaemia, idiopathic leucopenia, ulcerative colitis, derinatomyositis, scleroderma, mixed connective tissue disease, rheumatoid arthritis, irritable bowel syndrome, SLE, Hashimoto's disease, thyroiditis, Behcet's disease, coeliac disease/dermatitis herpetifortnis, and demyelinating disease.

In a further embodiment of the method for increasing production of an NFATc2-dependent cytokine in a subject in need of such treatment, the subject is afflicted with a hyperplastic condition or with a viral infection.

In certain aspects, the invention provides a method of preventing or reducing immune incompatibility in a subject in need thereof, the method comprising administering to the subject a therapeutically effective amount of an inhibitor RNA construct that decreases expression of NFATc2 or an NFATc2-regulated factor. An inhibitor RNA construct may also be referred to as an siRNA construct, including short double stranded RNAs and hairpin RNAs with a short region of internal complementarity. In a preferred embodiment, the NFATc2-regulated factor is a factor that is regulated by NFATc2 in cells of umbilical cord blood. Examples of such factors include: IL-3, IL-4, IL-5, IL-13, GM-CSF, IFN-γ, TNF-α, CD40L and MIP-1α.

The inhibitor construct may be generated by one skilled in the art using the methods and procedures provided by the present invention. For example, an siRNA inhibitor can be generated which targets IL-13 and administered in a therapeutically effective amount, through any of the methods described herein, to a subject to prevent or reduce immune incompatibility. In a preferred embodiment, the subject is a recipient of a transplant comprising a bone marrow transplant or a solid organ transplant.

In addition to the immune system disorders described above, methods for modulating NFATc2 disclosed herein may be used to affect essentially any process in which NFATc2 participates.

NFATc2 participates in angiogenesis. NFATc2 signaling is necessary for the proper formation of endothelial tubes and subsequent vessel formation, particularly in post-natal mammals. Accordingly, a method for increasing NFATc2 activity in a cell may be used to stimulate angiogenesis. In certain embodiments, modulators of NFATc2 may be used to treat or otherwise affect angiogenesis associated diseases and processes. For example, conditions in which it is desirable to inhibit angiogenesis (inhibit NFATc2 activity in endothelial cells by, for example, IFN-γ antagonist or NFATc2 pathway siRNA) include, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases (e.g., diabetic retinopathy, retinopathy of prematurity, macular degeneration, corneal graft rejection, neovascular glaucoma) retrolental fibroplasia, rubeosis; Osler-Webber Syndrome, telangiectasia; hemophiliac joints and angiofibroma. Examples of conditions in which it is desirable t stimulate angiogenesis (activate NFATc2 activity in endothelial cells by, for example, IFN-γ agonist) include wound granulation and wound healing; formation of coronary collateral vessels, angiogenesis in ischemic limbs, fracture healing, treatment of vascular problems in diabetics, especially retinal and peripheral vessels, and promotion of vascularization of cardiac muscle.

NFATc2 participates in the postnatal growth of skeletal muscle; NFATc2 deficiency is associated with the formation of small multinucleated muscle cells. Accordingly, modulators of NFATc2 may be used to modulate the skeletal muscle mass. Increased skeletal muscle mass may be desirable after muscle injury or in muscle wasting disorders, which may occur as a part of the aging process, as a result of inactivity or as a result of various disease processes or therapeutic agents.

NFATc2 participates in cartilage formation. NFATc2 downregulation is associated with excess cartilage formation due to increased chondrocyte cell proliferation and differentiation. Therefore, modulators of NFATc2 in mesenchymal stem cells and chondrocytes may be used to assist in regeneration of damaged cartilage in joints or other cartilaginous tissues (e.g., damage resulting from rheumatoid arthritis, physical injury, surgery, wear and tear, etc.).

Additional NFATc2 regulated process may be identified by analysis of NFATc2 heterozygous and homozygous knockout mice, although the differences in NFATc2 between mice and humans are such that further research in human cells is necessary to verify and validate results obtained in mice. Additional NFATc2-regulated processes may also be identified by analysis of various human cell types in vitro or in vivo.

3. Interferon Agonists and Antagonists

A. Agonists

The IFN-γ agonists used in the methods of the present invention comprise any agent or substance which increases the activity of the IFN-γ, or which directly or indirectly decreases IFN-γ signaling. The IFN-γ agonist may physically mimic an IFN-γ polypeptide or fragment thereof, such that the agonist mimics or increases IFN-γ signaling through its receptor. The IFN-γ agonist may comprise agents which increase signaling through the IFN-γ signaling pathway, including those which increase the activity of the IFN-γ receptor, the JAK1 and JAK2 kinases associated with the IFN-γ receptor, STAT proteins, and other of its intracellular signaling pathway (See Dorman and Holland, Cytokine Growth Factor Rev., 2000, 11, 321-333).

In one embodiment, the IFN-γ agonist comprises an IFN-γ polypeptide. IFN-γ and pharmaceutical formulations of IFN-γ have been substantially reported in the literature. See e.g., U.S. Pat. Nos. 5,198,212, 5,151,265, 5,132,110, 5,082,659, 5,082,658, 4,950,470, 4,929,443, 4,751,078, 4,723,000, 4,714,611, 4,696,899, and 4,686,284, which relate to IFN-γ containing compositions and the use thereof as pharmaceuticals. Also, numerous active analogs of IFN-γ have been reported in the patent and non-patent literature. See e.g., U.S. Pat. Nos. 5,096,705, 5,004,689, 4,898,931, 4,908,432, 4,921,698, 4,835,256, 4,832,959, and 4,758,656 which report active gamma interferon analogs.

In one embodiment, the IFN-γ agonist comprises a biologically active fragment of IFN-γ. In a further embodiment, the biologically active fragment of IFN-γ comprises a C-terminal peptide of IFN-γ. U.S. Pat. Nos. 6,120,762 and 5,770,191 describe C-terminal peptides of IFN-γ that are biologically active.

In another embodiment, the IFN-γ agonist comprises a modified form of interferon, such as those containing modifications which increase their stability or their biologically activity. The modification may comprise poly-ethylene glycol groups or polydextrin groups, such as those described in U.S. patent Publication US20030138403, hereby incorporated by reference in its entirety. Additional modification of IFN-γ polypeptides or fragments to be used a IFN-γ agonists on the methods of the present invention include, but are not limited to, palmatoyl modification (Thiam et al. (1998) Biochem Biophys Res Commun; 253(3):639-47).

In another embodiment, the IFN-γ agonist comprises a genetically modified form of IFN-γ polypeptide. Such modifications, include amino acid substitution, which increase the stability or activity of the IFN-γ polypeptide are desired. The IFN-γ agonist may comprise, for example, the IFN-γ polypeptides described in U.S. Pat. Nos. 6,300,474 and 6,299,870.

In another embodiment, the IFN-γ agonists comprise agents which boost the endogenous production of IFN-γ by a patient's T-cells or by natural killer (NK) cells. NK cells can be induced to secrete IFN-γ through activation of the KIR2DL4 receptor by an antibody or another agonist, for example, as described in International PCT Publication No. WO/0234290A2.

In another embodiment, the IFN-γ agonists comprise antibodies or fragments thereof. In a further embodiment, the antibodies bind to the IFN-γ receptor, which is comprised of two subunits, interferon gamma receptor 1 and 2, such that they activate the receptors, initiating the JAK-mediated phosphorylation cascade. In another embodiment, the antibody or antibody fragment used on the methods of the present invention comprise single-chain Fv fragments, chimeric antibodies, diabodies, triabodies, tetravalent antibodies, peptabodies and hexabodies.

As used herein, “antibody” means an immunoglobulin molecule comprising two heavy chains and two light chains and which recognizes an antigen. The immunoglobulin molecule may derive from any of the commonly known classes, including but not limited to IgA, secretory IgA, IgG and IgM. IgG subclasses are also well known to those in the art and include but are not limited to human IgG1, IgG2, IgG3 and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. Optionally, an antibody can be labeled with a detectable marker. Detectable markers include, for example, radioactive or fluorescent markers. The antibody may be a human or nonhuman antibody. The nonhuman antibody may be humanized by recombinant methods to reduce its immunogenicity in man. Methods for humanizing antibodies are known to those skilled in the art. As used herein, “monoclonal antibody,” also designated as mAb, is used to describe antibody molecules whose primary sequences are essentially identical and which exhibit the same antigenic specificity. Monoclonal antibodies may be produced by hybridoma, recombinant, transgenic or other techniques known to one skilled in the art.

The term “antibody” includes, but is not limited to, both naturally occurring and non-naturally occurring antibodies. Specifically, the term “antibody” includes polyclonal and monoclonal antibodies, and antigen-binding fragments thereof. Furthermore, the term “antibody” includes chimeric antibodies, wholly synthetic antibodies, and antigen-binding fragments thereof. Accordingly, in one embodiment, the antibody is a monoclonal antibody. In one embodiment, the antibody is a polyclonal antibody. In one embodiment, the antibody is a humanized antibody. In one embodiment, the antibody is a chimeric antibody. Such chimeric antibodies may comprise a portion of an antibody from one source and a portion of an antibody from another source.

In one embodiment, the portion of the antibody comprises a light chain of the antibody. As used herein, “light chain” means the smaller polypeptide of an antibody molecule composed of one variable domain (VL) and one constant domain (CL), or fragments thereof. In one embodiment, the portion of the antibody comprises a heavy chain of the antibody. As used herein, “heavy chain” means the larger polypeptide of an antibody molecule composed of one variable domain (VH) and three or four constant domains (CH1, CH2, CH3, and CH4), or fragments thereof. In one embodiment, the portion of the antibody comprises a Fab portion of the antibody. As used herein, “Fab” means a monovalent antigen binding fragment of an immunoglobulin that consists of one light chain and part of a heavy chain. It can be obtained by brief papain digestion or by recombinant methods. In one embodiment, the portion of the antibody comprises a F(ab′)₂ portion of the antibody. As used herein, “F(ab′)2 fragment” means a bivalent antigen binding fragment of an immunoglobulin that consists of both light chains and part of both heavy chains. It can be obtained by brief pepsin digestion or recombinant methods. In one embodiment, the portion of the antibody comprises a Fd portion of the antibody. In one embodiment, the portion of the antibody comprises a Fv portion of the antibody. In one embodiment, the portion of the antibody comprises a variable domain of the antibody. In one embodiment, the portion of the antibody comprises a constant domain of the antibody. In one embodiment, the portion of the antibody comprises one or more CDR domains of the antibody. As used herein, “CDR” or “complementarity determining region” means a highly variable sequence of amino acids in the variable domain of an antibody.

In another embodiment of the methods provided by the invention, the IFN-γ agonist or antagonist comprises a humanized antibody. As used herein, “humanized” describes antibodies wherein some, most or all of the amino acids outside the CDR regions are replaced with corresponding amino acids derived from human immunoglobulin molecules. In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody would retain a similar antigenic specificity as the original antibody.

Those IFN-γ agonists described herein which comprise polypeptides may be administered to a subject either directly as polypeptides or alternatively by introducing nucleic acids which encode the polypeptides. Such gene therapy methods may be used to introduce for example, an IFN-γ polypeptide or a humanized antibody which binds to either IFN-γ or to its receptor into a subject.

Various methods of transferring or delivering DNA to cells for expression of the gene product protein which can be easily adapted for the expression of an IFN-γ agonist/antagonist are disclosed in Gene Transfer into Mammalian Somatic Cells in vivo, N. Yang, Crit. Rev. Biotechn. 12(4): 335-356 (1992), which is hereby incorporated by reference. Gene transfer methods for gene therapy fall into three broad categories-physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA.

In a preferred embodiment, the DNA encodes a secreted form of the IFN-γ agonist/antagonist, such that lymphocytes can then be exposed to the secreted protein.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then re-implanted in the patient. In in vitro gene transfer, the transformed cells are cells growing in culture, such as tissue culture cells, and not cells particularly derived from a given patient. These “laboratory cells” are transfected, the transfected cells are selected and expanded for either implantation into a patient or for other uses. These cells can then secrete the IFN-γ agonist/antagonist in the patient. Depending on the half-life of the cells and the desired length of therapeutic treatment, this process of implanting transfected cells into the patient may be repeated as desired.

In one embodiment, the cells transfected with the DNA which encodes the IFN-γ agonist/antagonist are cells which are intended to be transplanted into a patient, such as cells from a bone marrow transplant or cells from a solid organ transplant. For example, cells from a bone marrow transplant can be transfected with an IFN-γ antagonist in vitro prior to grafting into a patient, or a solid organ can be transfused with the DNA encoding the IFN-γ agonist/antagonist in an appropriate transfecting composition prior to transplanting into the patient.

In vivo gene transfer involves introducing the DNA into the cells of the patient when the cells are already within the patient. Methods include using virally mediated gene transfer using a noninfectious virus to deliver the gene in the patient or injecting naked DNA into a site in the patient and the DNA is taken up by a percentage of cells in which the gene product protein is expressed. Additionally, the other methods described herein, such as use of a “gene gun,” may be used for in vitro insertion of IFN-γ agonist/antagonist DNA.

Chemical methods of gene therapy may involve a lipid based compound, not necessarily a liposome, to ferry the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the patient to provide the gene product from the inserted DNA.

Viral vectors have also been used to insert genes into cells using in vivo protocols. To direct tissue-specific expression of foreign genes, cis-acting regulatory elements or promoters that are known to be tissue specific can be used. Alternatively, this can be achieved using in situ delivery of DNA or viral vectors to specific anatomical sites in vivo. For example, gene transfer to blood vessels in vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs. If the IFN-γ agonist/antagonist is designed such that it is secreted from a cell, introduction of a DNA encoding the IFN-γ agonist/antagonist into endothelial cells may result in secretion of the IFN-γ agonist/antagonist.

Mechanical methods of DNA delivery include fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” and inorganic chemical approaches such as calcium phosphate transfection. Another method, ligand-mediated gene therapy, involves complexing the DNA with specific ligands to form ligand-DNA conjugates, to direct the DNA to a specific cell or tissue. Particle-mediated gene transfer methods were first used in transforming plant tissue. With a particle bombardment device, or “gene gun,” a motive force is generated to accelerate DNA-coated high density particles (such as gold or tungsten) to a high velocity that allows penetration of the target organs, tissues or cells. Particle bombardment can be used in in vitro systems, or with ex vivo or in vivo techniques to introduce DNA into cells, tissues or organs.

Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. This technique can be used in in vitro systems, or with ex vivo or in vivo techniques to introduce DNA into cells, tissues or organs.

Carrier mediated gene transfer in vivo can be used to transfect foreign DNA into cells. The carrier-DNA complex can be conveniently introduced into body fluids or the bloodstream and then site specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoportein/polylysine conjugate system for carrying DNA to hepatocytes for in vivo gene transfer.

The transfected DNA may also be complexed with other kinds of carriers so that the DNA is carried to the recipient cell and then resides in the cytoplasm or in the nucleoplasm. DNA can be coupled to carrier nuclear proteins in specifically engineered vesicle complexes and carried directly into the nucleus.

B. Antagonists

The IFN-γ antagonists used in the methods of the present invention comprise any agent or substance which decreases the activity of IFN-γ, or which directly or indirectly decreases IFN-γ signaling. The IFN-γ antagonist may physically bind to IFN-γ, to the IFN-γ receptor, or to other components of the IFN-γ signaling pathway such as the JAK1 and JAK2 kinases associated with the IFN-γ receptor, the STAT proteins, and therefore inhibit signal transduction. The IFN-γ antagonist, for example, might bind to IFN-γ or to its receptor and prevent an interaction between the two. Alternatively, the antagonists may reduce the expression level of the IFN-γ or of one of the components of the IFN-γ signaling pathway, thus antagonizing IFN-γ signaling activity.

In some embodiments, IFN-γ antagonists comprise RNA interference (RNAi) reagents to induce knockdown of IFN-γ or of a protein which transduces a IFN-γ signal, such as an IFN-γ receptor. RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of in some instances as few as 21 to 22 base pairs in length. Furthermore, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′,5′ oligoadenylate synthetase (2′,5′-AS), which synthesizes a molecule that activates Rnase L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

RNAi has been shown to be effective in reducing or eliminating the expression of a gene in a number of different organisms including Caenorhabditis elegans (see e.g. Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discemable to the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g. Expedite RNA phophoramidites and thymidine phosphoramidite (Proligo, Germany). Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

If IFN-γ is the target of the double stranded RNA, any of the above RNA species will be designed to include a portion of a nucleic acid sequence that hybridizes, under stringent and/or physiological conditions to the IFN-γ mRNA sequence, such as Genbank Accession No: NM_{—}000619. Likewise, if the target is the IFN-γ receptor, which comprises two subunits, interferon gamma receptor 1 (also known as IFNgammaR1, IFNGR1, IFN-gamma receptor alpha chain and cd119w) and interferon gamma receptor 2 (also known as IFNgammaR2, IFNGR2, IFN-gamma receptor-beta, IFN-gamma transducer 1, AF-1 and GAF), then any of the above RNA species will be designed to include a portion of a nucleic acid sequence that hybridizes, under stringent and/or physiological conditions to the corresponding mRNA sequences of either or both of the interferon gamma receptor subunits. U.S. patent application Ser. No. 09/843,377, published as US2003/0176371, describes methods and compositions for the antisense modulation of interferon gamma receptor 2 expression, the contents of which are incorporated herein by reference.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the invention.

The dsRNA oligonucleotides may be introduced into the cell by transfection with an heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). Further compositions, methods and applications of RNAi technology are provided in U.S. Pat. Nos. 6,278,039, 5,723,750 and 5,244,805, which are incorporated herein by reference.

Ribozyme molecules designed to catalytically cleave IFN-γ encoding mRNAs, or mRNAs encoding other members of the IFN-γ signaling pathway (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event.

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach (1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA. The present invention extends to ribozymes which hybridize to a sense mRNA encoding IFN-γ or an IFN-γ pathway member when an IFN-γ antagonist is desired.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme.

In a long target RNA chain, significant numbers of target sites are not accessible to the ribozyme because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (see Milner et al. (1997) Nat Biotechnol 15: 537-41; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the invention.

In certain embodiments, expression of the “target gene, whether it is NFATc2, an NFATc2-regulated gene, or an IFN-γ pathway member, may be inhibited by an inhibitor RNA that is a single-stranded RNA molecule containing an inverted repeat region that causes the RNA to self-hybridize, forming a hairpin structure (a so-called “hairpin RNA” or “shRNA”). shRNA molecules of this type may be encoded in RNA or DNA vectors. The term “encoded” is used to indicate that the vector, when acted upon by an appropriate enzyme, such as an RNA polymerase, will give rise to the desired shRNA molecules (although additional processing enzymes may also be involved in producing the encoded shRNA molecules). The expression of shRNAs may be constitutive or regulated in a desired manner.

A double-stranded structure of an shRNA is formed by a single self-complementary RNA strand. RNA duplex formation may be initiated either inside or outside the cell. Inhibition is sequence-specific in that nucleotide sequences corresponding to the duplex region of the RNA are targeted for genetic inhibition. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Because 100% sequence identity between the RNA and the target gene is not required to practice the present invention, the invention has the advantage of being able to tolerate sequence variations that might be expected due to genetic mutation, strain polymorphism, or evolutionary divergence. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. An endogenous RNA polymerase of the cell may mediate transcription of an shRNA encoded in a nucleic acid construct. The shRNA construct may also be synthesized by a bacteriophage RNA polymerase (e.g., T3, T7, SP6) that is expressed in the cell.

In another embodiment of the methods described herein, the IFN-γ antagonist is an antibody or fragment thereof which binds IFN-γ. Without intending to be bound by mechanism, the antagonistic antibody that binds IFN-γ may sterically prevent the binding of IFN-γ to the IFN-receptor and/or may decreases the half-life of IFN-γ by enhancing its degradation.

IFN-γ binding antibodies, such as those described in U.S. Pat. No. 6,350,860, hereby incorporated by reference, may be used. Additional antibody compositions directed at IFN-γ are described in U.S. patent application Ser. Nos. 10/243,197 and 09/972,656, the contents of which are incorporated by reference.

In another embodiment, the IFN-γ antagonist comprises antibodies or fragments thereof which bind to the IFN-γ receptor. In one embodiment, the antibody binding to the IFN-γ receptor causes the receptor to be internalized, prevents IFN-γ binding to the receptor, or prevents receptor activation upon IFN-γ binding. Thus, the antibody may be a competitive or a noncompetitive inhibitor of the binding reaction between IFN-γ and its receptor. In addition, an antibody which binds to the IFN-γ receptor and induces its internalization may also be used as an IFN-γ antagonist.

In another embodiment, the IFN-γ antagonist comprises a non-antibody IFN-γ-binding polypeptide. In a specific embodiment, the IFN-γ-binding polypeptide comprises a secreted viral protein, such as the protein B8R from the Vaccinia virus (Poxviridae), or a fragment thereof which retains the ability to bind IFN-γ (Alcami et al., J Virol. 69(8): (1999) 4633-9). Such viral proteins may titrate the IFN-γ and prevent its binding to the IFN-γ receptor.

In another embodiment, the non-antibody IFN-γ-binding polypeptide comprises a soluble fragment of the IFN-γ receptor, such as a fragment comprising the extracellular domain. Such polypeptides are described, for example, in U.S. Pat. Nos. 4,897,264, 5,221,789, and 5,578,707. The non-antibody IFN-γ-binding polypeptides described herein may be further modified to increase their stability when administered to a subject, such as fusing by fusing the IFN-γ-binding polypeptide to the Fc domain of human IgG or fusing the polypeptide to human serum albumin.

Additional IFN-γ antagonist are described in U.S. Pat. No. 6,558,661, the contents of which are hereby incorporated by reference.

In another embodiment, the IFN-γ antagonist comprises a small molecule or drug. In a specific embodiment, the small molecule or drug blocks the interaction between IFN-γ and its receptor. Such small molecules may be identified using common screening methods known to one skilled in the art.

4. Immunological Disorders

The invention additionally provides methods of preventing or reducing immune incompatibility in a subject in need thereof, the method comprising administering to the subject a therapeutically effective amount of an IFN-γ antagonist. In one embodiment, the subject has or is at risk for having graft versus host disease (GVHD). The methods described herein to treat or prevent HVGD may be applied to acute and to chronic GVHD. Acute GVHD typically occurs within the first three months following a transplant. Chronic GVHD occurs two or three months after a transplant and may include symptoms similar to autoimmune diseases and rashes, and may include liver, stomach and intestinal problems.

The methods of the invention are suitable for the prevention and treatment of GVHD in the course of bone marrow transplantation in patients suffering from diseases curable by bone marrow transplantation, including leukemias, such as acute lymphoblastic leukemia (ALL), acute nonlymphoblastic leukemia (ANLL), acute myelocytic leukemia (AML) and chronic myelocytic leukemia (CML), severe combined immunodeficiency syndromes (SCID), osteopetrosis, aplastic anemia, Gaucher's disease, thalassemia and other congenital or genetically-determined hematopoietic or metabolic abnormalities. The need for a bone marrow stem cell transplant arises because the only treatment that appears to have a chance of killing the disease in the host also kills the host's cellular immune system. Thus, the patient or host is treated to kill the target disease, and as a result of such treatment, the host's cellular immune system is also killed. Common methods of treatment include radiation treatment and chemotherapy, either alone or together, with or without accompanying surgery.

After such treatment, it is necessary to provide the patient with a means for regenerating the patient's immune system. The bone marrow or bone marrow hematopoietic stem cell transplant provides the basis for this immune system regeneration. The donor is treated to enrich his or her blood with bone marrow stem cells. The donor's blood is drawn and is centrifuged to separate the white blood cells which include the desired stem cells necessary to regenerate the host's immune system from the rest of the blood. The separated white blood cells will also include the donor's T-cells. The separated white blood cells form the transplant innoculum that is infused into the host. After infusion into the host, the infused or transplanted stem cells will seed the host's bone marrow and will differentiate into different blood cell types. This regeneration of the immune system, i.e., the production by the host of T-cells which can attack aberrant cells, such as infected cells, takes several weeks to several months.

In one embodiment, a therapeutically effective of an IFN-γ antagonist is administered to the subject under a suitable conditioning regimen, such as from day −2 prior to the transplantation day, and then for another 60-100, at least 60, days, after the transplantation day. As used herein, the term therapeutically effective amount means an amount of IFN-γ antagonist of the pharmaceutical composition or method that is sufficient to show a meaningful patient benefit, i.e., a reduction in the incidence or severity of acute or chronic graft-versus-host disease compared to that expected for a comparable group of patients not receiving the IFN-γ antagonist, as determined by the attending physician. When applied to an individual active ingredient administered alone, the term refers to that ingredient alone. When applied to a combination, the term refers to combined amounts of the active ingredients that result in the therapeutic effect, whether administered in combination, serially, or simultaneously. Ultimately, the attending physician will decide on the appropriate duration of and dosage of the IFN-γ treatment.

The methods described herein for treating or preventing HVGD are in some embodiments used in combination with other treatments. In one embodiment, a patient suffering or at risk of developing chronic GVHD can be treated with steroids such as cyclosporine, prednisone, and ozothioprine, or with cyclosporine and methotrexate, while at the same time be treated using one IFN-γ antagonists.

In one embodiment, the IFN-γ antagonist administered to a patient to prevent or treat GVHD is administered in a dosage where the GVHD is reduced but where it is not completely eliminated. A low level of GVHD is in some cases beneficial for the stem cell graft to colonize the patient bone marrow. Additional, the presence of active T-cells from a donor may help eliminate tumorigenic cells in a subject, such as a subject afflicted with leukemia.

The invention also provides a method of preventing graft versus host disease in a subject in need of such treatment, the method comprising contacting a transplant, prior to transplantation into the subject, with an IFN-γ antagonist, thereby preventing graft versus host disease in the subject. In one embodiment, a therapeutically effective amount of an IFN-γ antagonist is administered to the transplant innoculum prior to transplantation into the subject. In another embodiment, the transplant innoculum is depleted of T cells, such as by centrifugation, and the graft is then treated with IFN-γ antagonists prior to implantation into the patient or host.

The invention also provides methods of preventing or reducing immune incompatibility in a subject in need thereof, the method comprising administering to the subject a therapeutically effective amount of an IFN-γ antagonist, wherein the subject is a recipient of a solid organ transplant. In one embodiment, the organ transplant comprises a lung, heart, kidney, liver, or skin. The transplant may be HLA-matched or it may be unmatched.

In one embodiment, the solid organ is treated with an IFN-γ antagonist, such as by perfusion, prior to transplantation. In another embodiment, the IFN-γ antagonist is administered to the subject as described for treating or preventing GVHD, but wherein a therapeutically effective amount used refers to the amount of IFN-γ antagonist sufficient to show a meaningful patient benefit, i.e., a reduction in the incidence or severity of organ rejection compared to that expected for a comparable group of patients not receiving the IFN-γ antagonist, as determined by the attending physician.

Another aspect of the invention provides methods of preventing or reducing immune incompatibility in a subject in need thereof, the method comprising administering to the subject a therapeutically effective amount of an inhibitor RNA construct that decreases expression of NFATc2 or an NFATc2-regulated factor. The inhibitor RNA construct can be any of the variants described in the present invention. In one embodiment, the inhibitor is an siRNA. The inhibitory RNAi may be administered to the subject locally, such as at the site of an organ transplant, or alternatively it may be administered systemically, or both.

In one embodiment, the subject has or is at risk for having graft versus host disease, such as a recipient of a hematopoietic stem cell transplant. Hematopoietic stem cell transplant as used herein may comprises hematopoetic stem cells from an unrelated donor, umbilical vein hematopoetic stem cells, or peripheral blood stem cells.

In another embodiment, the subject has or is at risk for having graft rejection, such as a subject who is the recipient of a solid organ transplant. Transplants may be is HLA-matched or HLA-unmatched, and they may be allogenic.

In one embodiment, the NFATc2-regulated factor is selected from among the following: IL-3, IL-4, IL-5, IL-13, GM-CSF, IFN-γ, TNF-α, CD40L and MIP-1α. Inhibitors directed to any of these genes can be generated by a skilled artisan using the methods disclosed in the invention and common knowledge.

The invention also provides methods of treating an autoimmune disease in a subject in need thereof, the method comprising administering to the subject a therapeutically effective amount of an IFN-γ antagonist. In one embodiment, the autoimmune disease is selected from among the following: primary myxoedema, thyrotoxicosis, pernicious anaemia, autoimmune atrophic gastris, Addison's disease, IDDM, Goodpasture's syndrome, myasthenia gravis, sympathetic ophthalmia, MS, autoimmune haemolytic anaemia, idiopathic leucopenia, ulcerative colitis, derinatomyositis, sclerodenna, mixed connective tissue disease, rheumatoid arthritis, irritable bowel syndrome, SLE, Hashimoto's disease, thyroiditis, Behcet's disease, coeliac disease/dermatitis herpetifortnis, and demyelinating disease.

5. Assays for Evaluating NFATc2 Modulating Agents

Identification of a role of IFN-γ in regulating NFATc2 allows for evaluating and screening candidate NFATc2 modulating agents that may be used in various disorders, including GVHD, transplant rejection and other processes in which NFATc2 participates. In general, a method for evaluating an NFATc2 modulating agent comprises providing an IFN-γ agonist or antagonist and evaluating the effects of the IFN-γ agonist or antagonist on NFATc2 activity. Generally, a candidate agent that causes in increase in NFATc2 activity will be considered an activator of NFATc2, and a candidate agent that causes a increase in NFATc2 activity will be considered an inhibitor of NFATc2. NFATc2 activity may be assessed in cell culture or in an animal or other assay system. In a preferred embodiment, measuring an effect of the agent on an NFATc2 activity comprises measuring expression of NFATc2 or an NFATc2-regulated gene in an umbilical cord blood T cell culture. Examples of NFATc2-regulated genes include GM-CSF, CTLA-4, IFN-γ, IL-2Rα, IL-3, IL-4, IL-5, IL-13, TNF, CD40L, MIP-1α, p21, CCNA2 and CCNE2. Measuring NFATc2 activity may also include measuring a phenomenon that is highly correlated with NFATc2 activity, such as a shift in localization from the cytoplasm to the nucleus, or a shift in phosphorylation state.

A candidate agent may be a known IFN-γ agonist or antagonist, such as an IFN-γ polypeptide or active variant thereof, or a neutralizing anti-IFN-γ antibody. A method may also involve the generation and assessment of a new IFN-γ agonist or antagonist. New agonists or antagonists may be rationally designed, or may simply be new antibodies or new IFN-γ polypeptide variants. Providing an IFN-γ agonist or antagonist may comprise screening a plurality of agents to identify an agent having IFN-γ agonist or antagonist activity. The agonist or antagonist may be identified as interfering with or promoting IFN-γ signaling. The agonist or antagonist may be identified as interfering with or promoting the interaction between IFN-γ and a receptor. The agonist or antagonist may be identified as altering the expression level of IFN-γ expression. Other aspects of IFN-γ activity may also be used to develop assays for identifying agonists and antagonists. A wide variety of assays may be used for this purpose, including labeled in vitro protein-protein binding assays, electrophoretic mobility shift assays, interaction trap assay, immunoassays for protein binding, and the like.

A variety of assay formats will suffice and, in light of the present disclosure, those not expressly described herein will nevertheless be comprehended by one of ordinary skill in the art. Assay formats which approximate such conditions as formation of protein complexes, enzymatic activity, may be generated in many different forms, and include assays based on cell-free systems, e.g. purified proteins or cell lysates, as well as cell-based assays which utilize intact cells. Simple binding assays can also be used to detect agents which bind to IFN-γ. Such binding assays may also identify agents that act by disrupting the interaction between an IFN-γ polypeptide and an IFN-γ receptor. Agents to be tested can be produced, for example, by bacteria, yeast or other organisms (e.g. natural products), produced chemically (e.g. small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In a further embodiment, agents that bind to IFN-γ or an IFN-γ receptor may be identified by using an immobilized IFN-γ or an IFN-γ receptor protein, or portion thereof. In an illustrative embodiment, a fusion protein can be provided which adds a domain that permits the protein to be bound to an insoluble matrix. For example, GST-IFN-γ receptor fusion proteins can be adsorbed onto glutathione sepharose beads (Sigma Chemical, St. Louis, Mo.) or glutathione derivatized microtitre plates, which are then combined with a potential labeled binding agent and incubated under conditions conducive to binding. Following incubation, the beads are washed to remove any unbound agent, and the matrix bead-bound label determined directly, or in the supernatant after the bound agent is dissociated.

With an IFN-γ agonist or antagonist in hand, a variety of assays are available for assessing the effects of the candidate agent on NFATc2. Specific protocols for certain assays are provided in the Examples, below.

A method may comprise further assessments of the activator or inhibitor, such as evaluations of effects on in vivo or in vitro disease models. A preferred disease model is a model for graft versus host disease. An additional preferred disease model is a model for graft rejection. A method may further comprise evaluating the effect of the candidate agent on graft versus host disease or transplant rejection in an animal. A method may further comprise regulated clinical trials in volunteer human subjects.

6. Compositions, Formulations and Administration

The methods described herein comprise the administration of IFN-γ agonists or antagonists. In preferred embodiments, the agonist and antagonists described herein are formulated into pharmaceutical compositions. For example, the agonist and antagonists and their physiologically acceptable salts and solvates may be formulated for administration by, for example, by aerosol, intravenous, oral or topical route. The administration may comprise intralesional, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

An exemplary composition of the invention comprises an RNAi mixed with a delivery system, such as a liposome system, and optionally including an acceptable excipient. In a preferred embodiment, the composition is formulated for injection.

Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the agonists/antagonists of the invention can be formulated in liquid solutions, preferably in physiologically compatible buffers such as Hank's solution or Ringer's solution. In addition, the agonists/antagonists may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the agonists/antagonists for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The agonists/antagonists may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The agonists/antagonists may also be formulated in rectal compositions such as suppositories or retention enemas, e.g., containing conventional suppository bases such as cocoa butter or other glycerides.

In addition to the formulations described previously, the agonists/antagonists may also be formulated as a depot preparation. Such long acting formulations may be administered by implantation (for example subcutaneously or intramuscularly) or by intramuscular injection. Thus, for example, the agonists/antagonists may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the agonists/antagonists can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

Toxicity and therapeutic efficacy of the agents and compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agonists/antagonists which exhibit large therapeutic induces are preferred. While agonists/antagonists that exhibit toxic side effects may be used, care should be taken to design a delivery system that targets such agonists/antagonists to the site of affected tissue in order to minimize potential damage to uninfected cells and, thereby, reduce side effects.

The data obtained from the cell culture assays and animal studies can be used in formulating a range of dosage for use in humans. The dosage of such agonists/antagonists lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In one embodiment of the methods described herein, the effective amount of the agent is between about 1 mg and about 50 mg per kg body weight of the subject. In one embodiment, the effective amount of the agent is between about 2 mg and about 40 mg per kg body weight of the subject. In one embodiment, the effective amount of the agent is between about 3 mg and about 30 mg per kg body weight of the subject. In one embodiment, the effective amount of the agent is between about 4 mg and about 20 mg per kg body weight of the subject. In one embodiment, the effective amount of the agent is between about 5 mg and about 10 mg per kg body weight of the subject.

In one embodiment of the methods described herein, the agent is administered at least once per day. In one embodiment, the agent is administered daily. In one embodiment, the agent is administered every other day. In one embodiment, the agent is administered every 6 to 8 days. In one embodiment, the agent is administered weekly.

As for the amount of the compound and/or agent for administration to the subject, one skilled in the art would know how to determine the appropriate amount. As used herein, a dose or amount would be one in sufficient quantities to either inhibit the disorder, treat the disorder, treat the subject or prevent the subject from becoming afflicted with the disorder. This amount may be considered an effective amount. A person of ordinary skill in the art can perform simple titration experiments to determine what amount is required to treat the subject. The dose of the composition of the invention will vary depending on the subject and upon the particular route of administration used. In one embodiment, the dosage can range from about 0.1 to about 100,000 ug/kg body weight of the subject. Based upon the composition, the dose can be delivered continuously, such as by continuous pump, or at periodic intervals. For example, on one or more separate occasions. Desired time intervals of multiple doses of a particular composition can be determined without undue experimentation by one skilled in the art.

The effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The effective amount will be known to one of skill in the art; it will also be dependent upon the form of the compound, the size of the compound and the bioactivity of the compound. One of skill in the art could routinely perform empirical activity tests for a compound to determine the bioactivity in bioassays and thus determine the effective amount. In one embodiment of the above methods, the effective amount of the compound comprises from about 1.0 ng/kg to about 100 mg/kg body weight of the subject. In another embodiment of the above methods, the effective amount of the compound comprises from about 100 ng/kg to about 50 mg/kg body weight of the subject. In another embodiment of the above methods, the effective amount of the compound comprises from about 1 ug/kg to about 10 mg/kg body weight of the subject. In another embodiment of the above methods, the effective amount of the compound comprises from about 100 ug/kg to about 1 mg/kg body weight of the subject.

As for when the compound, compositions and/or agent is to be administered, one skilled in the art can determine when to administer such compound and/or agent. The administration may be constant for a certain period of time or periodic and at specific intervals. The compound may be delivered hourly, daily, weekly, monthly, yearly (e.g. in a time release form) or as a one time delivery. The delivery may be continuous delivery for a period of time, e.g. intravenous delivery. In one embodiment of the methods described herein, the agent is administered at least once per day. In one embodiment of the methods described herein, the agent is administered daily. In one embodiment of the methods described herein, the agent is administered every other day. In one embodiment of the methods described herein, the agent is administered every 6 to 8 days. In one embodiment of the methods described herein, the agent is administered weekly In one embodiment, the IFN-γ antagonist is administered to a patient extracorporeally. For example, an antagonist comprising an IFN-γ binding protein may be immobilized on a matrix, and blood or serum can be withdrawn from a patient and exposed to the matrix, such that IFN-γ in the blood or serum adheres to the matrix and is therefore removed from the blood or serum. The blood or serum can then be returned to the patient, effectively “dialyzing” the IFN-γ away from the patient. Such a method is described in U.S. Pat. No. 5,626,843, hereby incorporated by reference.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

Exemplification

The invention now being generally described, it will be more readily understood by reference to the following examples, which are included merely for purposes of illustration of certain aspects and embodiments of the present invention, and are not intended to limit the invention.

### EXPERIMENTAL PROTOCOLS FOR EXAMPLES 1-6

The following protocols were employed in performing the experiments described in Examples 1-6, below.

Cells

Human umbilical cord blood from full-term deliveries by vaginal or caesarean sections and adult peripheral blood from healthy donors was collected according to guidelines by the Institutional Review Board at University Hospitals of Cleveland. Mononuclear cells (MNC) were isolated by gradient centrifugation, as previously described (6) and used immediately fresh.

T Cell Stimulations

Total MNC were stimulated in bulk culture as previously described (6). Briefly, 2×10⁶ cells/ml were stimulated with 2 μg/ml of concanavalin A (ConA) (Sigma Chemical Co, St Louis, Mo.) in complete RPMI medium (Gibco BRL, Gaithersburg, Md.), containing 10% fetal bovine serum (Gibco BRL), 1 mM Sodium-pyruvate, 0.1 mM nonessential amino acids, 10 mM HEPES and 58 μM 2-mercaptoethanol (Sigma Chemical Co), in presence or absence of the manufacturer's recommended concentration of 1 μg/ml of cyclosporin A (CsA) (Sigma Chemical Co), as previously described (7). Where specified, IFN-γ (Roche, Indianapolis, Ind.) was added at indicated concentrations, or neutralizing anti-IFN-γ antibody (R&D Systems, Minneapolis, Minn.) was added at a concentration of 0.25-2 μg/ml. Purified T cells were stimulated as described (26), with plate-bound anti-CD3 (1 μg/ml) and soluble anti-CD28 (5 μg/ml) monoclonal antibodies. Elispot assays were performed as previously described (27). Briefly, ImmunoSpot plates M200 (Cellular Technology Limited, Cleveland Ohio) were coated with capture antibody for IFN-γ M700A-E (2 μg/ml; Endogen, Woburn, Mass.), washed and MNC were plated in complete RPMI medium with 5% ABO serum (Gemini Bioproducts, Calmasas, Calif.)+1% L-glutamine, at 3×10⁵ per well. Cells were stimulated with irradiated 3×10⁵ T-cell-depleted MNC from a healthy adult or cord blood. T cells were depleted with RosetteSep (StemCell Technologies, Vancouver, Canada), according to manufacturer's directions. After 24 h of stimulation, plates were washed and incubated over night with secondary biotinylated detection antibodies against IFN-γ M701 (0.5 μg/ml; Endogen, Woburn, Mass.). Streptavidin-HRP conjugate (Dako Corp., Carpenteria Calif.) was added and the spots were visualized using the HRP-substrate AEC (Pierce Pharmaceuticals, Rockford Ill.), and subjected to image analysis on a Series 1 ImmunoSpot Image Analyzer (Cellular Technology, Cleveland, Ohio) specifically designed for automated evaluation of ELISPOTs.

T Cell Purification

T cells were purified from MNC by depletion of monocytes, B and NK cells, using a cocktail of monoclonal antibodies including: CD11b, CD16, CD19 and CD56 (Pharmingen) followed by anti-IgG magnetic bead depletion (Dynal, Lake Success, N.Y.), as previously described (6).

Depletion of Monocytes

MNC from UCB were depleted of monocytes by using monoclonal antibody against CD11b (Pharmingen), followed by anti-IgG magnetic bead (Dynal) depletion as above, or by using anti-CD14 microbeads (Miltenyi Biotech Inc., Auburn, Calif.) and AutoMACS (Miltenyi Biotech Inc.), according to manufacturer's protocol.

Western Blot Analysis

At indicated time points of stimulation, T cells were purified from the MNC and extracted as previously described (6). Briefly, cells were lysed in 20 mM Tris pH 7.6, 50 mM KCl, 400 mM NaCl, 1 mM EDTA, 1% Triton-X, 20% glycerol, 1 mM DTT, 0.5 mM phenylmethylsulfonyl fluoride (PMSF), 1 μg/ml pepstatin, 2 μg/ml leupeptin, 2 μg/ml aprotinin and 10 mM Na₂MoO₄. 7 μg of protein lysate was immunodetected with a mix of monoclonal antibodies directed against NFATc2 (Transduction Laboratories, Lexington, Ky.) and β-actin (Sigma Chemical Co). NFATc2 and β-actin band intensities were quantified by densitometry scanning.

Flow Cytometry

Intracellular IFN-γ staining was performed as described previously (6, 7). Briefly, MNC were stimulated for the indicated time points, for the last 6-8 h in the presence of 5 μg/ml of Brefeldin A (Sigma Chemical Co), and permeabilized. 1×10⁶ cells were stained for 30 minutes with the following antibodies: anti-IFN-γ-FITC or -PE (R&D Systems), anti-CD3-APC (Pharmingen), and anti-CD69-PE (Pharmingen) or -PerCP (Becton Dickinson, San Jose, Calif.) and the corresponding isotype controls (Pharmingen, Becton Dickinson). 20,000-80,000 events were acquired on a calibrated Elite ESP flow cytometer (Coulter, Miami, Fla.) and data were analyzed with WinList software (Verity Software House Inc, Topsham, Minn.). For intracellular staining of NFATc2, the same monoclonal antibody clone as used in immunoblotting was custom FITC-conjugated (Transduction Laboratories) and used at a titrated concentration of 1 μg/10⁶ cells. NFATc2-specificity of the conjugated antibody was verified by competition experiments with unconjugated anti-NFATc2 antibody, unconjugated unrelated murine IgG and unconjugated anti-NFATc and anti-NFAT3 antibodies. For competition of FITC-conjugated anti-NFATc2 antibody binding, cells were incubated prior to the staining with the FITC-conjugated antibody, with either the unlabeled anti-NFATc2 antibody, or with NFATc1 (Santa Cruz Biotechnology, Santa Cruz, Calif.) or NFATc3 (Santa Cruz Biotechnology) antibody or unconjugated unrelated murine IgG (Sigma Chemical Co.), in 4-fold excess for 30 minutes. Cells were washed and then stained with FITC-conjugated anti-NFATc2 antibody for additional 30 minutes, washed and 20,000 events were acquired on a calibrated Elite ESP flow cytometer (Coulter). While competition with unconjugated anti-NFATc1, NFATc3 and unrelated murine IgG did not result in reduced binding of FITC-conjugated anti-NFATc2 antibody, competition with the unconjugated anti-NFATc2 resulted in significantly reduced binding of the conjugated antibody.

Statistical Analysis

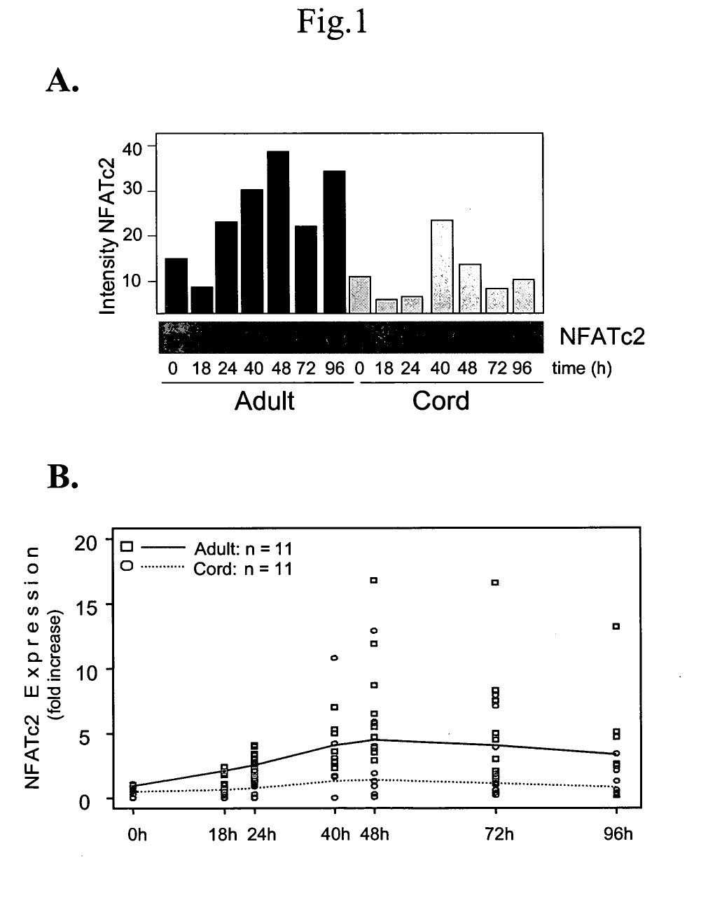

NFATc2-expression data was fitted using PROC MIXED in SAS (Version 8.0, Cary, N.C.) with random intercept and unstructured covariance structure (FIG. 1B). The slopes before 50 hours were estimated, and tested by two-sided T-test (28).

### Example 1

### Reduced Expression of NFATc2 Protein in UCB T Lymphocytes During Prolonged Primary Stimulation

Our previous studies in UCB T cells suggested a correlation between low NFATc2 and IFN-γ expression during the first 40 h of stimulation (6). To verify that upregulation of NFATc2 expression in UCB T lymphocytes is reduced and not delayed in time, mononuclear cells (MNC) from adult peripheral blood and umbilical cord blood were stimulated for up to 96 hours, after which T lymphocytes were purified and NFATc2 protein levels measured, as previously described. At later time points, strong variability in NFATc2 protein expression was observed between the individual samples studied, particularly in the adult controls. Peak expression ranged between 40-72 h, in both cord and adult T lymphocytes. Thereafter, NFATc2 protein expression decreased in both adult and UCB, but remained above basal levels, in the adult (FIG. 1A). However, notwithstanding the individual variability of NFATc2 protein expression upon stimulation, overall NFATc2 protein expression remained significantly reduced in UCB T lymphocytes, at all time points, compared to the adult analyzed within the same experiment. The fold-increase in NFATc2 protein expression plotted over time was notable for increased NFATc2 protein expression in adult T lymphocytes, with estimated slope 0.212 (p<0.0001), whereas fold-increase of NFATc2 expression in UCB, relative to adult expression, was marginal with estimated slope 0.09 (p=0.08) (FIG. 1B).

### Example 2

### In Adult T Lymphocytes, Upregulation of NFATc2 is IFN-γ-Dependent

Late upregulation of NFATc2 in adult T cells, as well as reduced and late upregulation in UCB T lymphocytes, suggested dependence on the presence of intermediary regulatory cytokines and/or proteins with reduced expression by UCB. As stimulation of primary T cells with concanavalin A (ConA) in the presence of IFN-γ was found to result in a substantial increase in loading-control mRNAs such as β-actin, GADPH and HPRT, thus rendering analysis of specific increases in NFATc2 mRNA expression difficult, Applicants chose to analyze NFATc2 expression more precisely by intracellular staining and flow cytometric analysis, in gated CD3⁺ cells.

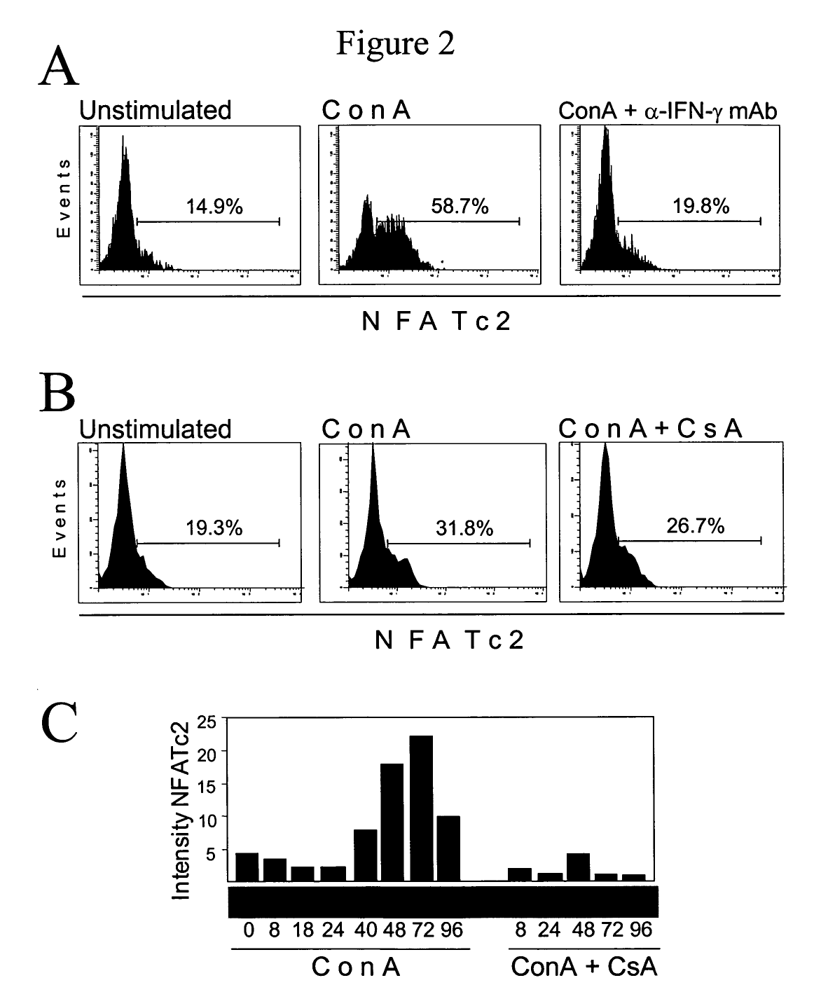

As IFN-γ production during T cell stimulation is not impaired in adult T lymphocytes, IFN-γ blocking experiments were performed to determine the effect on NFATc2 levels in adult T lymphocytes. By flow cytometry, percentages of NFATc2-expressing T cells varied between adult individuals, and were noted to increase upon stimulation, and to range from 24-81% after 24-48 hours of stimulation (n=9), comparable to the individual variability in NFATc2 expression patterns observed in Western blot analyses. When IFN-γ secreted during stimulation of adult T cells was blocked with neutralizing anti-IFN-γ antibody, upregulation of NFATc2 protein expression was blunted and did not increase significantly above basal expression levels (FIG. 2A). Inhibition was observed with different anti-IFN-γ antibody clones, and effectiveness of blunting of NFATc2 expression was found variable from individual to individual, in accordance with varying IFN-γ production from individual to individual.

Since IFN-γ production and other proteins regulated by NFAT can be inhibited by the immunosuppressive drug Cyclosporin A (CsA) (29) via inhibition of calcineurin, NFATc2 expression was measured in adult T lymphocytes after stimulation in the presence of CsA. As shown in FIG. 2B, NFATc2 protein upregulation in adult T cells was inhibited in the presence of CsA during stimulation. Inhibition of NFATc2 protein upregulation in the presence of CsA was confirmed by Western blot analysis, demonstrating the same blunting of NFATc2 protein upregulation (FIG. 2C). Taken together, these results indicate that NFATc2 expression is dependent in part on IFN-γ secreted during primary stimulation of adult T lymphocytes, and that by blocking secreted IFN-γ in the environment, NFATc2 mRNA and protein upregulation are reduced.

### Example 3

### Reduced NFATc2 Upregulation in UCB T Lymphocytes Can be Increased by Addition of IFN-γ

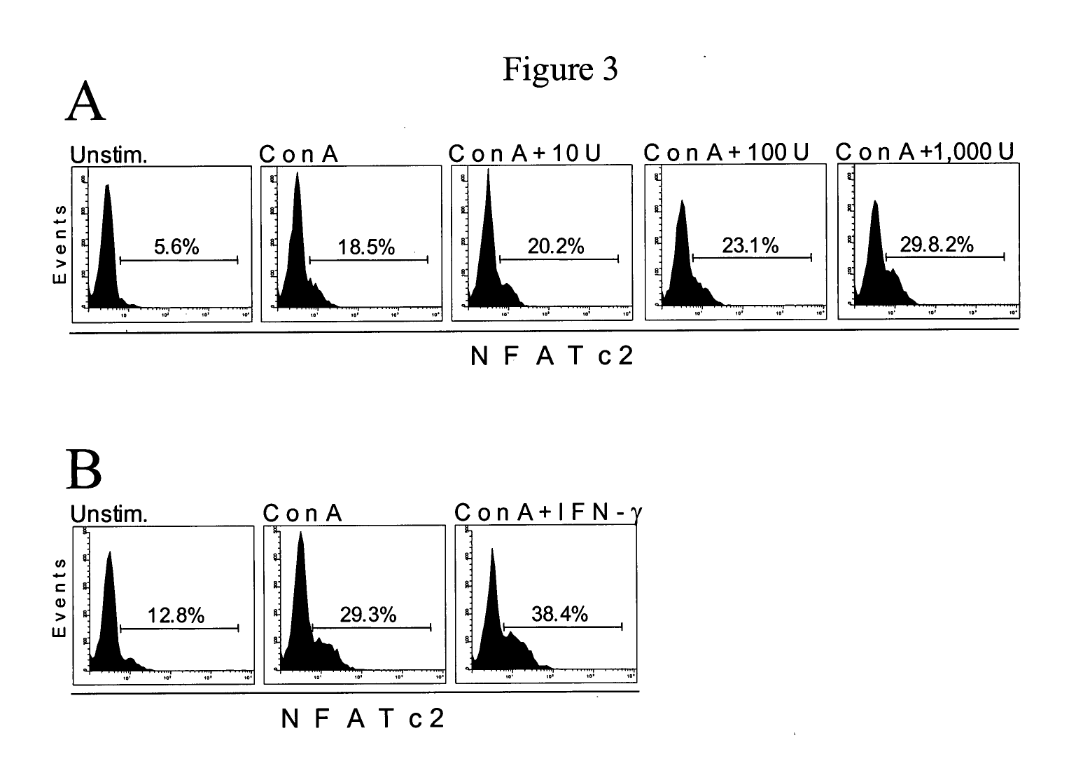

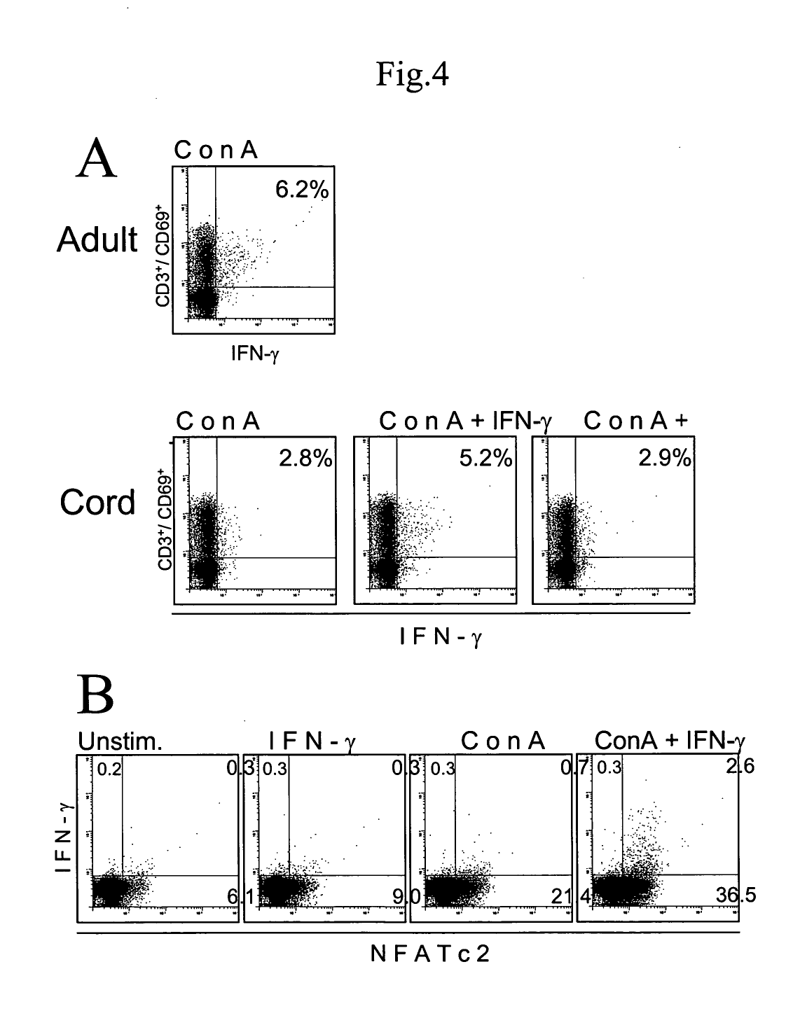

As UCB T lymphocytes have been shown to express reduced amounts of IFN-γ during primary stimulation (5, 6), studies were performed to determine whether adding exogenous IFN-γ could increase NFATc2 upregulation in UCB T lymphocytes. When UCB T lymphocytes were stimulated in the presence of increasing doses of added IFN-γ (10-1,000 U/ml), NFATc2 protein expression increased in a dose dependent manner (FIG. 3A). In the absence of stimulation, treatment with IFN-γ alone resulted in NFATc2 levels intermediate between unstimulated and stimulated T lymphocytes (FIG. 4B, lower right quadrant). In adult T lymphocytes, addition of exogenous IFN-γ also resulted in further upregulation of NFATc2 (FIG. 3B). These results support the hypothesis that upregulation of NFATc2 during stimulation is dependent in part on the presence of IFN-γ, and that reduced upregulation of NFATc2 in UCB T lymphocytes may be due to impaired IFN-γ production by UCB T lymphocytes.

### Example 4

### Rescue of IFN-γ Expression in UCB T Lymphocytes After IFN-γ-Induced Upregulation of NFATc2

In the NFATc2 gene-deleted mouse model, severely reduced IFN-γ expression was observed in homozygous animals, intermediate expression in heterozygotes, and normal expression in wild-type animals (18). Applicants therefore hypothesized that by increasing NFATc2 expression with added IFN-γ during stimulation, deficient IFN-γ expression by UCB T lymphocytes might be rescued. Exogenous IFN-γ was added during stimulation and cytoplasmic IFN-γ expression in UCB T lymphocytes was measured. When exogenous IFN-γ was added during stimulation of UCB T lymphocytes, percentages of IFN-γ-expressing T lymphocytes rose close to that measured in adult T lymphocytes, after only 24 h of stimulation (FIG. 4A). Effective IFN-γ rescue was observed titrating down to a dose of 50 U/ml of exogenous IFN-γ and was observed in over 20 cord blood units tested. This rescue effect was specific for IFN-γ, as addition of TNF-A or IL-2 did not result in upregulation of IFN-γ expression (FIG. 4A, ConA+TNF-A). Importantly however, the rescue-effect on IFN-γ expression in UCB T lymphocytes depended on upregulation of NFATc2, as shown by dual staining for NFATc2 and IFN-γ(FIG. 4B). IFN-γ expression was only observed in NFATc2 co-expressing T cells. Moreover, only the combination of ConA stimulation and addition of exogenous IFN-γ resulted in strong increases of NFATc2 expression and concomitant IFN-γ expression within the same cells, while treatment with IFN-γ alone did not stimulate IFN-γ expression and only slightly increased NFATc2 expression. Stimulation with ConA alone resulted in increase in NFATc2 upregulation, with only a slight increase in IFN-γ expression.

### Example 5

### Increase of IFN-γ Expression by IFN-γ in UCB T Cells is Dependent on the Presence of Antigen-Presenting Cells

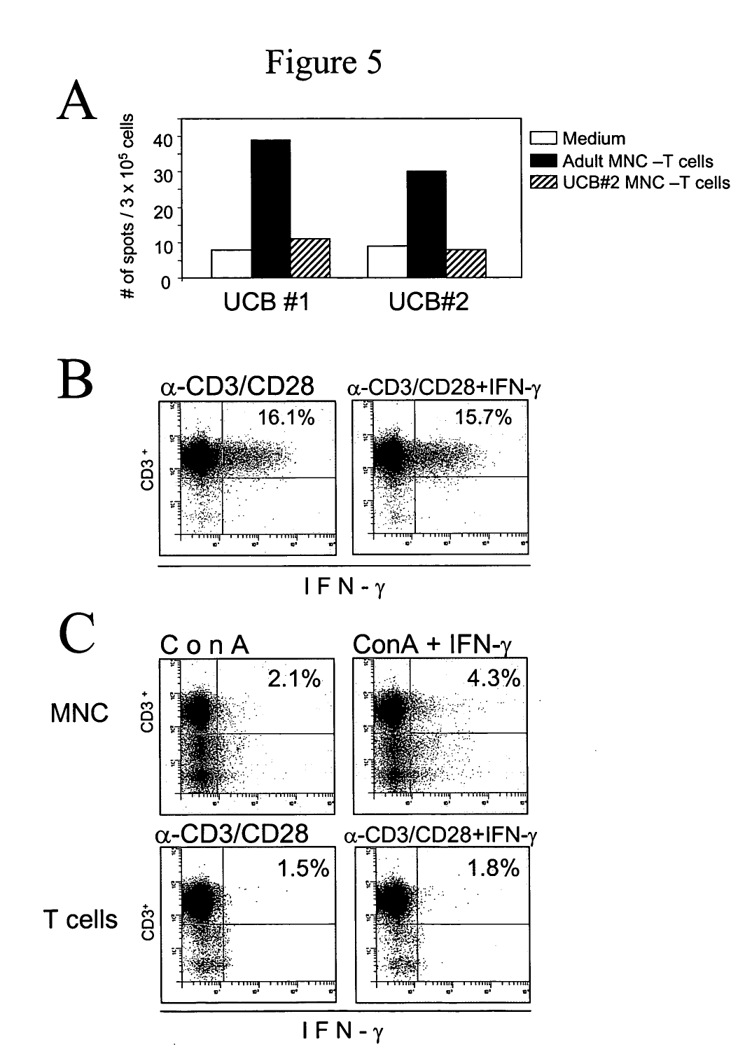

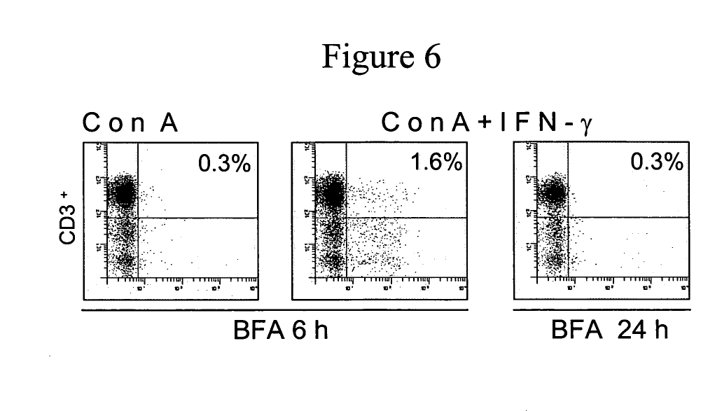

The above-described rescue of IFN-γ expression was observed in a stimulation setting where T cells were stimulated within whole MNC fractions, containing monocytes, B and NK cells. Since murine neonatal and human UCB antigen-presenting cells (APCs) have been described impaired in co-stimulation, adhesion molecules and cytokine expression, (30-34), Applicants asked the question whether the observed rescue effect by IFN-γ is exerted directly on the UCB T cells or via APCs present within the whole MNC preparation. When analyzing IFN-γ production upon allogeneic stimulation (MLR) by Elispot assays, Applicants found that UCB T cells produced higher amounts of IFN-γ when stimulated with adult T-cell-depleted MNC as stimulators (FIG. 5A, solid bars), than upon stimulation with T-cell-depleted MNC from an second, allogeneic cord (FIG. 5A, hatched bars), underscoring a deficiency in co-stimulation for T-cell IFN-γ production by UCB APCs.

### Example 6

When purified T lymphocytes from adult or UCB were stimulated with ConA alone, not surprisingly, very low IFN-γ expression was observed in adult and almost no IFN-γ expression in UCB T cells. When IFN-γ was added during stimulation of UCB T cells, no IFN-γ rescue effect was observed. To provide co-stimulation, purified T cells were stimulated with plate-bound anti-CD3 and soluble anti-CD28 mAb, in presence or absence of IFN-γ. While adult T cells expressed, as expected, high levels of IFN-γ (FIG. 5B), no increase in IFN-γ expression was observed upon addition of exogenous IFN-γ. Purified UCB T cells expressed only very low amounts of IFN-γ upon stimulation with anti-CD3/CD28 (FIG. 5C, T cells), and, unlike our observations of UCB T cell responses in the presence of accessory cells (FIG. 5C, MNC), reduced IFN-γ expression in isolated T cells from UCB could not be rescued by the addition of exogenous IFN-γ (FIG. 5C, α-CD3/CD28+IFN-γ). These results suggest that IFN-γ-dependent upregulation of IFN-γ expression in UCB and adult T cells is mediated by accessory cells, possibly through IFN-γ-mediated upregulation of co-stimulatory genes that may be deficient in UCB. Replacing IFN-γ by co-stimulation with soluble anti-CD28 during ConA stimulation of bulk MNC did however not result in rescue of IFN-γ production by UCB T cells, despite equivalent CD28 expression on UCB T lymphocytes (35).

Applicants next depleted UCB MNC of monocytes and stimulated in presence or absence of IFN-γ (See table 1 below).

TABLE 1
Depletion of monocytes reduces rescue effect of IFN-γ.

| | | 1 | 2 | 3 | 4 |
|---|---|---|---|---|---|
| MNC | ConA | 0.96% | 0.78% | 0.96% | 1.05% |
| MNC | ConA + IFN-γ | 1.38% | 1.41% | 1.74% | 1.83% |
| MNC − CD11b | ConA + IFN-γ | 0.89% | 0.92% | 1.30% | N.D. |
| MNC − CD14 | ConA + IFN-γ | N.D. | N.D. | N.D. | 1.07% |
| This table summarized 4 independent experiments. | | | | | |
| MNC from UCB were stimulated in absence or presence of IFN-γ, after depletion of either CD11b (experiments # 1, 2, 3) or CD14 (experiments # 4) and IFN-γ expression was assessed after 24 h by intracellular staining. A minimum of 25,000 gated CD3⁺ cells were acquired, | | | | | |
| # and numbers indicate percentages of CD3⁺ cells positive for IFN-γ staining. | | | | | |

N.D.: Not done.

When whole MNC from UCB were stimulated with ConA, an average of 0.9% (±0.1, n=4) of T cells produced IFN-γ. Upon stimulation in the presence of added IFN-γ, the percentage of IFN-γ producing cells increased to 1.6% (±0.2, n=4). This increase was blunted when MNC were depleted prior to stimulation, to an average of 1.0% (±0.2, n=4, p<0.005) of IFN-γ producing T cells. Depletion of B cells and NK cells did not result in a significant loss of the IFN-γ-mediated rescue effect, consistent with the hypothesis that the IFN-γ-mediated rescue effect is mediated by monocytes.

When the secretory pathway blocker Brefeldin A (BFA) (36) was added immediately and for the whole length of ConA stimulation, no upregulation of IFN-γ could be observed in UCB T cells (FIG. 5C, BFA 24 h). Upregulation of IFN-γ expression could only be observed when BFA was added for the last 6 hours of stimulation (BFA 6 h), thus allowing for prior secretion and/or surface expression of factors by APCs.

Taken together, the data presented in Examples 1-6 indicate that reduced IFN-γ expression observed in human UCB T cells is attributable at least in part to reduced expression of NFATc2. Moreover, supplementing human cell cultures with exogenous IFN-γ during stimulation increased both deficient NFATc2 and IFN-γ expression in UCB T cells. This IFN-γ-mediated rescue effect was not observed when isolated T cells were stimulated, nor when T cells were stimulated in presence of NK and B cells, but in absence of monocytes. While the precise mechanism of action remains uncertain (and relatively unimportant for clinical applications disclosed herein), these data suggest that the IFN-γ-mediated rescue effect requires the presence of APCs, which are generally present in vivo. No rescue effect was observed when protein export was inhibited, suggesting dependency on secretion of cytokines and/or upregulation of co-stimulatory molecules by the APCs.

Applicants have previously reported that CD45RA single positive T lymphocytes from adult peripheral blood expressed NFATc2 protein levels equivalent to the entire T cell population, and that CD45RO⁺UCB T lymphocytes express the same reduced NFATc2 levels as CD45RA single positive UCB T cells. This suggests that a lack of NFATc2 expression in the unstimulated UCB T lymphocyte is an intrinsic property rather than a trait of the predominantly “naive” CD45RA⁺ T cell population in UCB (6). Indeed, naive CD45RA⁺ UCB T lymphocytes may have distinct properties from the “naïve” peripheral adult T cell. Our results point to the intriguing possibility that in the adult, the CD45RO-negative, non-memory T cell is exposed to varying levels of cytokines in the periphery, including Th1 lymphocyte or NK cell-derived IFN-γ, produced during ongoing persistent low-level pathogen exposure. This may result in maintenance of a basal level of NFATc2 protein expression, providing the naive peripheral adult T cell with the requirements for rapid induction of NFATc2-dependent immunomodulatory genes upon stimulation, including the IFN-γ gene itself. Consistent with this hypothesis, Applicants observed a persistent drop in NFATc2 expression during the first hours of culture of adult T cells (FIG. 1A), suggesting response to withdrawal of cytokines. Moreover, addition of exogenous IFN-γ during stimulation of adult peripheral T cells further increased NFATc2 expression.

Our results pointing to a positive feedback loop between NFATc2 and IFN-γ evoke the possibility that severely reduced NFATc2 protein expression in the neonatal T lymphocyte may create a window of opportunity for appropriate T cell “maturation” during neonatal immune system ontogeny. One may hypothesize that in the absence of significant levels of NFATc2 protein, the neonatal T lymphocyte could remain unresponsive to benign environmental antigens that do not elicit proinflammatory cytokines such as IFN-γ, or could acquire responsiveness to pathogens encountered within the context of “danger” (37, 38). In the latter case, the presence of IFN-γ produced by the innate immune system, e.g. NK cells, dendritic cells (39, 40), or macrophages (41) in the vicinity, could increase NFATc2 expression during T cell stimulation, and thus not only allow for IFN-γ upregulation itself, but also upregulation of other NFATc2-dependent genes important in amplifying T cell-mediated immune responses. Potential NFATc2 upregulation by NK-cell or dendritic/macrophage-derived IFN-γ would furthermore link innate signals to the adaptive, antigen-specific T cell response, as strongest NFATc2 upregulation was only observed in the presence of T cell receptor triggering, added IFN-γ and presence of APCs (FIG. 4, 5).

Cellular mechanism underlying the impact of IFN-γ on NFATc2 and IFN-γ expression in T cells is unclear at this point. Our observation of requirement of APCs and moreover dependence on the secretory pathway, to mediate the observed IFN-γ-rescue effect on UCB T cells, suggests that IFN-γ induces upregulation of soluble factors or expression of surface molecules by the APCs, which then in turn stimulate NFATc2 and IFN-γ expression in T cells. It is interesting to note here that IL-12 has been shown to be upregulated in the presence of IFN-γ during endotoxin-stimulation of macrophages (42) and furthermore that IL-12 stimulates T cells to upregulate IFN-γ expression (43). This pathway is however deficient in UCB, as IL-12 production by LPS-stimulated MNC from UCB has been described severely reduced, compared to adult (44). By adding exogenous IL-12 to the stimulation conditions, reduced IFN-γ expression by UCB MNC could be increased. Interestingly, CB MNC were more sensitive than adult cells to IL-12 stimulation with respect to IFN-γ production (44), a phenomenon Applicants also observed, when adding IFN-γ to increase NFATc2 and IFN-γ expression. Our results thus point towards deficiencies both in the UCB T lymphocyte and APC population, resulting in reduced IFN-γ production during stimulation of UCB T cells.

In allogeneic UCB stem cell transplantation, while UCB T cell hyporesponsiveness, particularly reduced NFATc2/IFN-γ expression, may be one underlying mechanism of reduced GVHD observed after transplantation, it may also underlie higher infection-related morbidity and mortality observed after UCB transplantation (45). In both mouse and human, an IFN-γ Th1 response has been shown to be crucial for antibacterial and antiviral immunity, as well as for T cell cytotoxicity (46). Our results therefore provide clinical applications for modulating NFATc2 expression in emerging donor-derived T lymphocytes after stem cell transplantation, by impacting on IFN-γ levels with either IFN-γ neutralizing antibodies (or other IFN-γ antagonists) or by administered IFN-γ (or other IFN-γ agonists), in attempt to modulate the T-cell response after UCB transplantation.

The following references are numbered so as to correspond with the reference numbers in Examples 1-6 and the relevant experimental protocols.
1. Kurtzberg J, Laughlin M, Graham M L, Smith C, Olson J F, Halperin E C, Ciocci G, Carrier C, Stevens C E, Rubinstein P: Placental blood as a source of hematopoietic stem cells for transplantation into unrelated recipients. N Engl J Med 335:157-166, 1996
2. Rubinstein P, Carrier C, Scaradavou A, Kurtzberg J, Adamson J, Migliaccio A R, Berkowitz R L, Cabbad M, Dobrila N L, Taylor P E, Rosenfield R E, Stevens C E: Outcomes among 562 recipients of placental-blood transplants from unrelated donors. N. Engl. J. Med. 339:1565-1577, 1998
3. Rocha V, Wagner J J, Sobocinski K, Klein J, Zhang M, Horowitz M, Gluckman E: Graft-versus-host disease in children who have received a cord-blood or bone marrow transplant from an HLA-identical sibling. Eurocord and international bone marrow transplant registry working committee on alternative donor and stem cell sources. N Engl J Med 342:1846-1854, 2000
4. Laughlin M J, Barker J, Bambach B, Koc O N, Rizzieri D A, Wagner J E, Gerson S L, Lazarus H M, Cairo M, Stevens C E, Rubinstein P, Kurtzberg J: Hematopoietic engraftment and survival in adult recipients of umbilical-cord blood from unrelated donors. N Engl J Med 344:1815-1822, 2001
5. Chalmers IMH, Janossy G, Contreras M, Navarrete C: Intracellular cytokine profile of cord and adult blood lymphocytes. Blood 92:11-18, 1998
6. Kadereit S, Mohammad S, Miller R, Daum Woods K, Listrom C, McKinnon K, Alali A, Bos L, Iacobuci M, Sramkoski M, Jacobberger J, Laughlin M: reduced NFAT1 protein expression in human umbillical cord blood T lymphocytes. Blood 94:3101-3107, 1999
7. Kadereit S, Kozik M, Junge G, Miller R, Slivka L, Bos L, Daum-Woods K, Sramkoski R, Jacobberger J, Laughlin M: Cyclosporin A Effects During Primary And Secondary Activation Of Human Umbilical Cord Blood T Lymphocytes. Exp. Hematol. 29:903-909, 2001
8. Ferrara J L M, Krenger W: Graft-versus-host disease: The influence of type 1 and type 2 T cell cytokines. Transfusion Medicine Reviews 12:1-17, 1998
9. Klingebiel T, Schlegel P G: GVHD: Overview on pathophysiology, incidence, clinical and biological features. Bone Marrow Transplant 21:S45-S49, 1998
10. Pan L, Delmonte J J, Jalonen C K, Ferrara J L: Pretreatment of donor mice with granulocyte colony-stimulating factor polarizes donor T lymphocytes toward type-2 cytokine production and reduces severity of experimental graft-versus-host disease. Blood 86:4422-4429, 1995
11. Yang Y G: The role of interleukin-12 and interferon-gamma in GVHD and GVL. Cytokines Cell Mol Ther 6:41-46, 2000
12. Rao A, Luo C, Hogan P G: Transcription factors of the NFAT family: Regulation and Function. Ann. Rev. Immunol. 15:707-747, 1997
13. Campbell P, Pimm J, Ramassar V, Halloran P: Identification of a calcium-inducible, cyclosporine sensitive element in the IFN-gamma promoter that is a potential NFAT binding site. Transplantation 61:933-939, 1996
14. Feske S, Muller J M, Graf D, Kroczek R A, Drager R, Niemeyer C, Baeuerle P A, Peter H H, Schlesier M: Severe combined immunodeficiency due to defective binding of the nuclear factor of activated T cells in T lymphocytes of two male siblings. Eur J Immunol 26:2119-2126, 1996
15. Sica A, Dorman L, Viggiano V, Cippitelli M, Ghosh P, Rice N, Young H A: Interaction of NF-kappaB and NFAT with the interferon-gamma promoter. J Biol Chem 272:30412-30420, 1997
16. Sweetser M, Hoey T, Sun Y, Weaver W, Price G, Wilson C: The roles of nuclear factor of activated T cells and Ying-Yang 1 in activation-induced expression of the interferon-g promoter in T cells. J. Biol. Chem. 273:34775-34783, 1998
17. Kiani A, Roa A, Aramburu J: Manipulating immune responses with immunosppressive agents that target NFAT. Immunity 12:359-372, 2000
18. Hodge M R, Ranger A M, de la Brousse F C, Hoey T, Grusby M J, Glimcher L H: Hyperproliferation and dysregulation of IL-4 expression in NF-ATp-deficient mice. Immunity 4:397-405, 1996
19. Kiani A, Garcia-Cozar F J, I. H, Laforsch S, Aebischer T, Ehninger G, Rao A: Regulation of interferon-gamma gene expression by nuclear factor of activated T cells. Blood 98:1480-1488, 2001
20. McCaffrey P G, Luo C, Kerppola T K, Jain J, Badalian T M, Ho A M, Burgeon E, Lane W S, Lambert J N, Curran T, Verdine G L, Rao A, Hogan P G: Isolation of the cyclosporin-sensitive T cell transcription factor NFATp. Science 262:750-754, 1993
21. Lyakh L, Ghosh P, Rice N R: Expression of NFAT-Family proteins in normal human T cells. Mol Cell Biol 17:2475-2484, 1997
22. Crabtree G, Olson E: NFAT signaling: choreographing the social lives of cells. Cell 109:S67-79, 2002
23. Northrop J P, Ho S N, Chen L, Thomas D J, Timmerman L A, Nolan G P, Admon A, Crabtree G R: NF-AT components define a family of transcription factors targeted in T-cell activation. Nature 369:497-502, 1994
24. Diehl S, Chow C, Weiss L, Palmetshofer A, Twardzik T, Rounds L, Serfling E, Davis R, Anguita J, Rincon M: Induction of NFATc2 expression by interleukin 6 promotes T helper type 2 differentiation. J Exp Med 196:39-49, 2002
25. Cron R, Bort S, Wang Y, Brunvand M, Lewis D: T cell priming enhances IL-4 gene expression by increasing nuclear factor of activated T cells. J. Immunol. 162:860-870, 1999
26. Miller R E, Fayen J D, Mohammad S F, Stein K, Kadereit, S., Daum Woods K, Sramkoski R M, Jacobberger J W, Templeton D, Shurin S B, Laughlin M J: Reduced CTLA-4 Protein and Messenger RNA Expression in Umbilical Cord Blood T Lymphocytes. Exp Hematol 30:738-744, 2002
27. Helms T, Boehm B O, Asaad R J, Trezza R P, Lehmann P V, Tary-Lehmann M: Direct visualization of cytokine-producing recall antigen-specific CD4 memory T cells in healthy individuals and HIV patients. J Immunol 164:3723-3732, 2000
28. Verbeke G, Molenberghs G, editors. Linear Mixed Models in Practice: A SAS-Oriented Approach. New York: Springer; 1997.
29. Quesniaux V: Immunosuppressants: tools to investigate the physiological role of cytokines. Bioessays 15:731-739, 1993
30. Forsthuber T, Yip H C, Lehmann P V: Induction of TH1 and TH2 immunity in neonatal mice. Science 271:1728-1730, 1996
31. Ridge J P, Fuchs E J, Matzinger P: Neonatal tolerance revisited: turning on newborn T cells with dendritic cells. Science 271:1723-1726, 1996
32. Krampera M, Tavecchia L, Benedetti F, Nadali G, Pizzolo G: Intracellular cytokine profile of cord blood T-, and NK-cells and monocytes. Haematologica 85:675-679, 2000
33. Liu E, Tu W, H.K. L, Lau Y L: Changes of CD14 and CD1a expression in response to IL-4 and granulocyte-macrophage colony-stimulating factor are different in cord blood and adult blood monocytes. Pediatr Res 50:184-189, 2001
34. Varis I, Deneys V, Mazzon A, De Bruyere M, Comu G, Brichard B: Expression of HLA-DR, CAM and co-stimulatory molecules on cord blood monocytes. Eur J Haematol 66:107-114, 2001
35. Sato K, Nagayama H, Takahashi TA: Aberrant CD3- and CD28-mediated signaling events in cord blood T cells are associated with dysfunctional regulation of Fas ligand-mediated cytotoxicity. J Immunol 162:4464-4471, 1999
36. Dinter A, Berger E, G.: Golgi-disturbing agents. Histochem Cell Biol 109:571-590, 1998
37. Gallucci S, Matzinger P: Danger signals: SOS to the immune system. Curr. Opin. Immunol. 13:114-119, 2001
38. Matzinger P: An innate sense of danger. Semin. Immunol. 10:399-415, 1998
39. Ohteki T, Fukao T, Suzue K, Maki C, Ito M, Nakamura M, Koyasu S: Interleukin 12-dependent interferon gamma production by CD8alpha+lymphoid dendritic cells. J Exp Med 189:1981-1986, 1999
40. Hochrein H, Shortman K, Vremec D, Scott B, Hertzog P, O'Keeffe M: Differential production of IL-12, IFN-alpha, and IFN-gamma by mouse dendritic cell subsets. J Immunol 166:5448-5455, 2001
41. Puddu P, Fantuzzi L, Borghi P, Varano B, Rainaldi G, Guillemard E, Malorni W, Nicaise P, Wolf S F, Belardelli F, Gessani S: IL-12 induces IFN-gamma expression and secretion in mouse peritoneal macrophages. J Immunol 159:3490-3497, 1997
42. Chensue S W, Ruth J H, Warmington K, Lincoln P, Kunkel S L: In vivo regulation of macrophage IL-12 production during type 1 and type 2 cytokine-mediated granuloma formation. J Immunol 155:3546-3551, 1995
43. O'Garra A: Cytokines induce the development of functionally heterogeneous T helper cell subsets. Immunity 8:275-283, 1998
44. Lee S M, Suen Y, Chang L, V B, Qian J, Indes J, Knoppel E, van de Ven C, Cairo M S: Decreased interleukin-12 (IL-12) from activated cord versus adult peripheral blood mononuclear cells and upregulation of interferon-gamma, natural killer, and lymphokine-activated killer activity by IL-12 in cord blood mononuclear cells. Blood 88:945-954, 1996
45. Hamza N S, Lisgaris M V, Yadavalli G K, Fu P, Lazarus H M, Koc O N, Salata R A, Laughlin M J: Infectious complications after unrelated HLA-mismatched allogeneic umbilical cord blood transplantation (UCBT) in adults. Blood 98:204a, 2001
46. Dorman S E, Holland S M: Interferon-gamma and interleukin-12 pathway defects and human disease. Cytokine Growth Factor Rev 11:321-333, 2000
47. Cleveland W S: Robust locally weighted regression and smoothing scatterplots. J Amer Statist Assoc 74:829-836, 1979

### EXPERIMENTAL PROTOCOLS FOR EXAMPLES 7-15

Cells

With informed consent, human umbilical cord blood (UCB) from scheduled cesarean sections and adult peripheral blood (AB) were collected and mononuclear cells (MNC) purified as previously described. (Kadereit S, Mohammad S F, Miller R E, et al. Blood. 1999;94:3101-3107; Miller R E, Fayen J D, Mohammad S F, et al. Exp Hematol. 2002;30:738-744. For gene expression analysis using the HG-U133A&B GeneChip (Affymetrix, Santa Clara, Calif.), MNC from 7 independent UCB units and 7 control adults were depleted of CD14⁺ cells using CD14 microbeads (Miltenyi Biotech, Bergisch Gladbach, Germany) and CD4⁺ T-cells were selected using CD4 microbeads (Miltenyi). All cell separations were performed using AutoMACS magnetic cell sorter (Miltenyi). For hybridization to the HG-U95A GeneChip, MNC from 5 independent UCB units and 5 control adults were of depleted CD14⁺ cells using Dynabeads (Dynal, Lake Success, N.Y.) followed by positive selection of CD4⁺ T-cells using magnetic beads and manual columns (Miltenyi).

T-Cell Stimulation

For the HG-U133A&B gene array analysis, purified T-cells from each individual were divided into three equal populations. One population (0 hr) was extracted for RNA immediately, while the remaining two populations were stimulated with plate-bound anti-CD3 mAb (Hit3a, Pharmingen, San Diego, Calif.) at 5 μg/mL in PBS and with soluble anti-CD28 mAb (CD28.2, Pharmingen) at 5 μg/mL in RPMI-1640 (Gibco-BRL, Gaithersburg, Md.) with 10% fetal bovine serum (FBS). Cells were stimulated in 48-well plates with 1×10⁶ cells per well. Stimulated cells were harvested at 6 and 16 hours and RNA extracted immediately. For studies using the HG-U95A GeneChip, cells were extracted at 0 hour or stimulated as described above for 16 hours and RNA extracted immediately.

RNA Extraction and Pool Preparation

At the indicated time points, total RNA was isolated using TRIzo1 reagent per manufacturer's protocol (GibcoBRL). Purity was verified using electrophoresis and spectophotometry and samples were stored at −80° C. To obtain sufficient mRNA and reduce inter-individual genetic variability, equal amounts of RNA obtained from multiple individual UCB and AB donors was pooled for study at each time point. Initially, RNA from 5 UCB and 5 control adults were utilized for hybridization to the U95A microarray. This experiment was then repeated, using RNA pools from additional 7 UCB and 7 adult donors, with the exception of the adult 0 h RNA pool that contained RNA from 6 adults. In the replicate experiment, gene expression in the pooled samples was interrogated on the U133A&B microarray, and the same RNA samples were employed to validate observed changes in expression by RNase protection assays.

Purity Assessment and Confirmation of Surface Expression by Flow Cytometry

For purity assessment, cells were surface stained with fluorochrome-conjugated mAbs including CD3, CD4, CD8, CD14, CD19, CD56 (Pharmingen), and corresponding isotype controls. Confirmatory studies of surface expression were performed with mAbs against CD40L (Immunotech, Marseille, France), CXCR4, and CTLA-4 (Pharmingen). In each experiment, purified T-cells from 1 UCB and 1 adult control were stimulated as above and stained for CXCR4, CD40L, and CTLA-4 expression at 20 h. This was repeated four times. Fluorescence of more than 10,000 events was acquired on an LSR flow cytometer (Coulter, Miami, Fla.) and data were analyzed using WinList (Verity Software House Inc, Topsham, Minn.). Purity of CD4⁺/CD3⁺ ranged from 95-99% with the majority >97%.

Gene Expression Analysis

Gene expression was interrogated at the indicated time points using Affymetrix HG-U95A and HG-U133A&B expression microarrays (Affymetrix). In the initial experiment, RNA pools from 5 adult and 5 UCBs were hybridized to U95A microarrays. In the second experiment, Applicants used the newly released HG-U133A&B chip set, which contains 44,763 oligonucleotide probe sets for approximately 33,000 genes, to interrogate expression in additional 7 UCB units and 7 adult controls. It was decided to switch from HG-U95A to HG-U133A&B microarrays, since the HGU133A&B array set contains refined, more robust probe pairs and since probe sets to interrogate NFATc2 expression, lacking on the U95A chip, were added to this new generation of microarrays.

Pooled total RNA from each time point was used to prepare biotinylated target cRNA, with minor modifications from the manufacturer's recommendations (http://www.affymetrix.com/support/technical/manual/expression_manual.affx). Briefly, 8 μg of total RNA were used to generate first-strand cDNA by reverse transcription using a T7-linked oligo (dT) primer. After second-strand cDNA synthesis, in vitro transcription was performed with biotinylated UTP and CTP (Enzo Diagnostics), resulting in the generation of biotinylated cRNA that is approximately 100-fold amplified above the initial quantity of starting material. The target biotinylated cRNA generated from each time point was processed as per manufacturer's recommendation using an Affymetrix GeneChip Instrument System. Briefly, spike transcript controls and 15 μg of fragmented cRNA were added to a hybridization cocktail. This mixture was hybridized to the expression microarray by incubation at 45° C. overnight. Arrays were then washed and stained with streptavidin-phycoerythrin, before being scanned on an Affymetrix GeneChip scanner. After scanning, array images were assessed by eye to confirm scanner alignment and the absence of significant bubbles or scratches on the chip surface. 3′/5′ ratios for GAPDH and beta-actin were confirmed to be within acceptable limits (0.82-1.07), and BioB spike controls were found to be present on all chips, with BioC, BioD and CreX also present in increasing intensity. Finally, each image was scaled to a target intensity of 1500 and the scaling factors for all arrays were confirmed to be within acceptable limits (4.6-7), as were background and noise.

Data Analysis

For both the HG-U95A and HG-U133A&B arrays, the fluorescent intensity of each probe was quantified using Microarray Analysis Suite version 5.0 (MAS 5.0) software (Affymetrix). The expression level of a single mRNA, defined as the Signal, was determined by the MAS 5.0 software, which uses a weighted average fluorescence intensity difference obtained among the 11-20 probe pairs that interrogate the expression of each individual gene. This software also makes a detection call [Present (P), Marginal (M) or Absent (A)] for each gene or probe set, based on the consistency of the performance of the individual probe pairs, the hybridization above background, and the signal to noise ratio.

Two-way comparisons of the microarray data were also performed using the MAS 5.0 software. Specifically, changes in gene expression between UCB and adult samples were evaluated at each time point [i.e. UCB vs AB at each time point]. These comparisons in MAS 5.0 provided additional data including the Signal Log Ratio (fold change presented in logarithmic form) and the “Change Call” [Increased (I), Decreased (D), Marginally Increased (MI), Marginally Decreased (MD) or No Change (NC)] for each gene being interrogated. The data was then imported into a Microsoft Excel spreadsheet.

To identify genes that exhibited differences in expression between UCB and adult, the data sets were trimmed in Excel using the following inclusion criteria. For a gene or a probe set to be included in this trimmed data set, it had to display i) a Present Call (P) in at least one of the compared samples, and ii) a Change Call≠No Change (NC), and iii) a ≧2-fold difference in expression between the two compared samples. Additional annotation data was incorporated into the data set using the Affymetrix web-based analysis tool NetAffx.

GeneSpring Analysis

The signals displayed for the genes in each sample included in the HG-U133A&B trimmed data set, were imported into GeneSpring software version 5.1 (Silicon Genetics, San Carlos, Calif.). To present the relative expression for a given gene or probe set in each sample, the measured signal for each probe set was divided by the median of its measurements in all samples. If the median of the raw values was below 10 then each measurement for that gene or expressed sequence tag (EST) was divided by 10.

One-way hierarchical clustering with Pearson correlation analyses and minimum distance of 0.001 was employed to order genes in the trimmed data set for the time course carried out in the UCB and adult samples. The resulting gene tree incorporated all the genes/ESTs that had met inclusion criteria from the UCB v AB comparisons at any of the three time points (0, 6 and 16 hr primary stimulation).

RNase Protection Assay

To confirm the array data, expression of selected genes was analyzed in the same RNA pools that were hybridized to the HG-U133A&B arrays, using the RiboQuant multiprobe RNase protection (RPA) kits (Pharmingen). RPA kits included: hCK1, hStress and hCK2b. Replicate assays were performed according to the manufacturer's protocol using ³²P dUTP, on 5 μg of RNA; two times each for the hCK1 and hStress probe sets, and three times for the hCK2b probe set.

Multiplexed Cytokine Measurements

CD4⁺ T-cells were purified and each 1 UCB and AB sample was stimulated as described above for 20 hours. Supernatants were collected and analyzed in duplicate by a multiplexed cytokine array using fluorescent microspheres (Ref 27: Earley M C, Vogt R F, Jr., Shapiro H M, et al. Report from a workshop on multianalyte microsphere assays. Cytometry. 2002;50:239-242) at the Roswell Park Cancer Institute Flow Cytometry laboratory for the following cytokines and chemokines: IL-1β, IL-2, IL-3, IL-4, IL-5, IL-8, IL-10, IL-13, GM-CSF, MIG, MIP-1α and RANTES. This experimental set-up was repeated twice.

### Example 7

### Consistency of Microarray Expression Data

Two independent studies were carried out. The first examined gene expression in UCB and AB CD4⁺ T-cells at 0 h and 16 h of primary stimulation with anti-CD3 and anti-CD28, using the HG-U95A microarray. The second study assessed gene expression at 0, 6, and 16 h of stimulation using the HG-U133A&B microarray set. Data obtained from the 2 experiments was independently trimmed and analyzed using the same software and same inclusion criteria. After data analysis and trimming, the HG-U133A&B microarray data was compared to the dataset generated by the initial HG-U95A array experiment. The two array datasets were queried as follows: first, each dataset was queried for similar gene name, as annotated using the Netaffx annotations for each chip. At 0 hour, 129 probe sets had the same gene name. Of these, only 4 probes (3.1%) showed conflicting data, i.e. called Decreased on U95A and Increased on U133A&B. At 16 hour, 178 probe sets had the same gene name of which only 1 probe (0.56%) showed conflicting data. Next, the dataset was queried for similar Affymetrix probe ID numbers. This query revealed that at both 0 and 16 hours 100% showed corroboration of data between the U95A and U133A&B arrays. While data from the new HG-U133 microarray set cannot be directly compared to the HG-U95 microarray, our comparison however strongly suggests excellent reproducibility between these two microarray generations and thus robustness of the new HG-U133A&B microarrays. Further data mining was therefore carried out with the data from the HG-U133A&B microarrays.

### Example 8

### Results of Data Mining

Genes or probe sets meeting the inclusion criteria of at least a 2-fold difference between comparison groups at any of the three time points were subjected to one-way hierarchical clustering (FIG. 12A). This analysis revealed that UCB and AB exhibit distinct gene expression profiles at each time point, with more genes and ESTs exhibiting reduced expression in UCB than AB at each time point.

The final numbers of probe sets remaining after the inclusion analyses are shown in FIG. 12B. At 0 hour, 852 probe sets met the criteria for inclusion, while at 6 and 16 hour 1611 probe sets and 1187 probe sets, respectively, met the criteria. Importantly, the number of differentially expressed probe sets increased after stimulation, with the largest difference at 6 hours. The Venn diagram in FIG. 12B shows the overlap in these probe sets. 258 probe sets met the criteria for inclusion at all 3-time points. 141 probe sets met inclusion criteria at 0 and 6 hours; 337 at 6 and 16 hours; while only 77 probe sets were determined to be significant at 0 and 16 hours but not 6 hours.

### Example 9

### NFAT-Dependent Gene Expression

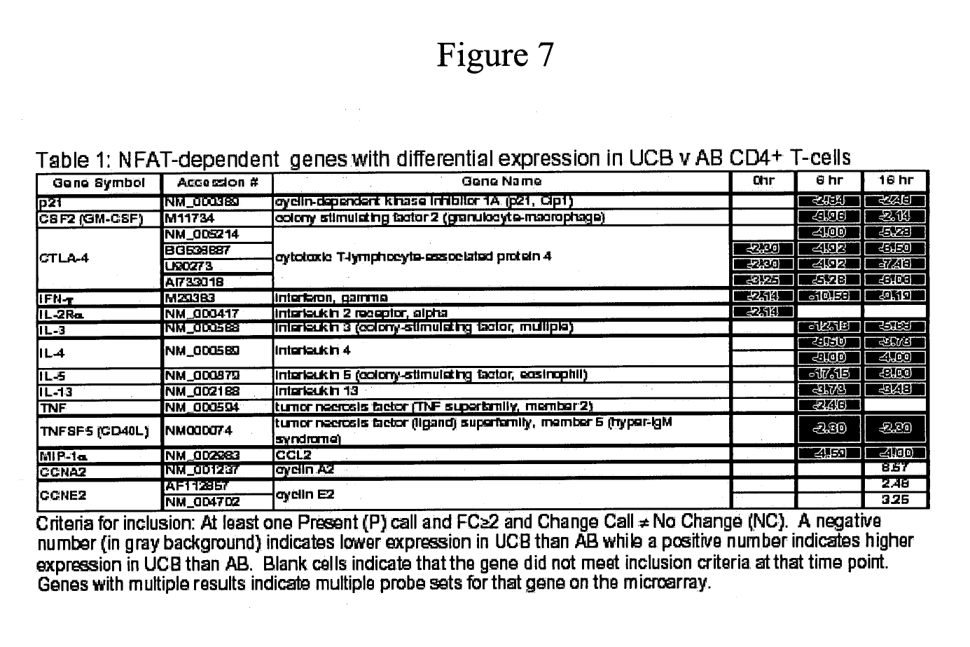

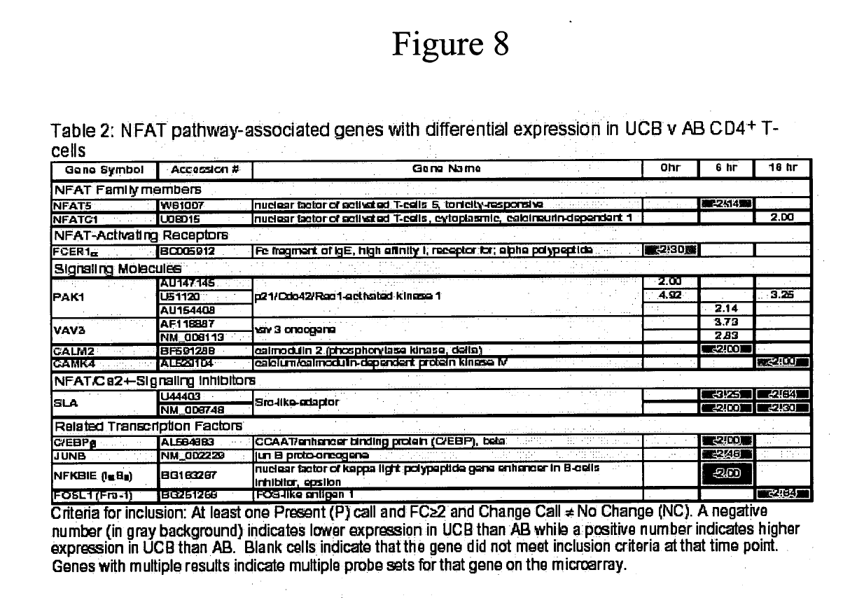

The NFAT pathway is crucial for expression of inflammatory cytokines and other immunomodulatory proteins as evidenced by NFATc2-gene deleted mice. (Hodge et al. Immunity. 1996;4:397-405) As NFATc2 is expressed at reduced levels in UCB (Kadereit et al. Blood. 1999;94:3101-3107), Applicants queried the microarray dataset for genes known to be dependent on NFATc2 and genes involved in the NFAT pathway (FIGS. 7 and 8). (Crabtree G. Cell. 1999;96: 611-614; Rao et al. 1997;15:707-747; Macian et al. EMBO J. 2000;19:4783-4795; Hodge et al. Immunity. 1996;4:397-405; Crabtree et al. Cell. 2002;109 Suppl:S67-79; Caetano et al. Faseb J. 2002;16:1940-1942) All known NFATc2-dependent genes on the HG-U133A&B microarray which met inclusion criteria, exhibited lower expression in UCB than AB CD4⁺ T lymphocytes, with the exception of the cyclins A2 and E2, which exhibited higher expression in UCB than AB (FIG. 7). The majority of differences were seen during stimulation, with the exception of CD25 (IL-2Rα) which only showed differential expression at 0 hr. NFATc2-dependent genes including IL-3, IL-4, IL-5, IL-13, GM-CSF, IFN-γ, TNF-α, CD40L, and MIP-1α demonstrated lower expression in UCB T lymphocytes after 6 and 16 hours of stimulation. CTLA-4 expression was lower in UCB than in AB at all three time points. Several cyclins including A2, B1, E, and F, have been shown to be over-expressed in NFATc2 gene-deleted murine lymphocytes after stimulation.29 Consistent with this report, our dataset revealed that cyclin A2 and cyclin E2 show greater expression in UCB than AB at 16 hr of stimulation.

### Example 10

### NFAT-Pathway Associated Gene Expression

Of the NFAT family members, only NFAT5 and NFATc1 showed differential expression comparing UCB and AB. Surprisingly, Applicants did not detect NFATc2 mRNA in either UCB, or in adult CD4+ T lymphocytes at any time point, in either of the 2 NFATc2 probe sets. Specifically, very low signals were detected by both probe sets, and were determined Absent in each sample by the MAS 5.0 algorithm. This inability to detect NFATc2 could result from the intrinsic low levels of this message and/or possibly reflect a lack of sensitivity of the 2 NFATc2 probe sets. In this regard, in previous RT-PCR experiments, which logarithmically amplify the message being evaluated, as compared to linear amplification of cRNA for hybridization to microarrays, NFATc2 mRNA could not be reproducibly detected at 0 h but could be detected consistently at 16 h in adult. NFAT5 (also known as Tonicity Enhancer Binding Protein) showed decreased expression in UCB at 6 hours of stimulation. Although originally described as a response to hypertonicity, it has been shown that NFAT5-dependent transcription can be induced by T-cell receptor (TCR)-dependent signaling events and is necessary for optimal T-cell development (Trama Jet al. J Immunol. 2000;165:4884-4894).

Although UCB T lymphocytes up-regulated NFAT5 expression by 6 hours of stimulation, this upregulation was attenuated when compared to the response seen in AB T lymphocytes. Expression fell to similar levels in both UCB and AB after 16 hours of stimulation. NFATc1 (NFAT2), which has been previously reported to be up-regulated within 3 hours of stimulation,31 was notably higher in UCB than AB at 16 hours. As UCB cells express low constitutive levels of NFATc2, the increase in NFATc1 mRNA at 16 hr stimulation may indicate a compensatory mechanism. Interestingly, examination of known NFAT-pathway genes, revealed that only PAK1 and VAV3 demonstrated higher expression in UCB compared to AB cells. CALM2 and CAMKIV (both calmodulin-related genes) demonstrated lower expression in UCB at 6 and 16 hours, respectively. SLAP, an NFAT/Ca2+ signaling inhibitor, exhibited lower expression in UCB after stimulation at both time points. Three related transcription factors also showed differential expression with C/EBPβ and JunB showing lower expression in UCB compared to AB at 6 hours, and Fosl1 (Fra-1) demonstrating lower expression by UCB CD4+ T lymphocytes at 16 hours.

### Example 11

### Cytokine and Cytokine Receptor mRNA Expression

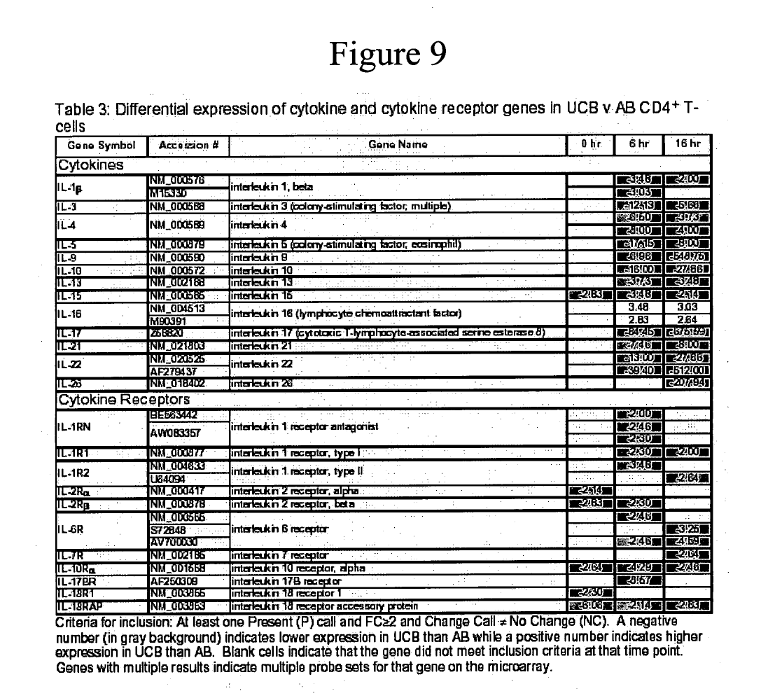

As cytokines and cytokine receptors play a crucial role in allogeneic inflammatory responses of T lymphocytes, Applicants queried the array data to investigate the expression of these genes in UCB compared to AB at all time points. Fourteen cytokine and ten cytokine receptor genes showed differential expression between UCB and AB CD4+ T lymphocytes at least one of the three time points (FIG. 9). Of these, all except IL-16 showed lower mRNA expression in UCB CD4+ T lymphocytes with the majority of the differences seen during stimulation. IL-16 expression, as detected by two probe sets, was 3.48-fold and 2.83-fold at 6 hr and 3.03-fold and 2.64-fold at 16 hr higher in UCB than AB.

### Example 12

### Chemokine and Chemokine Receptor mRNA Expression

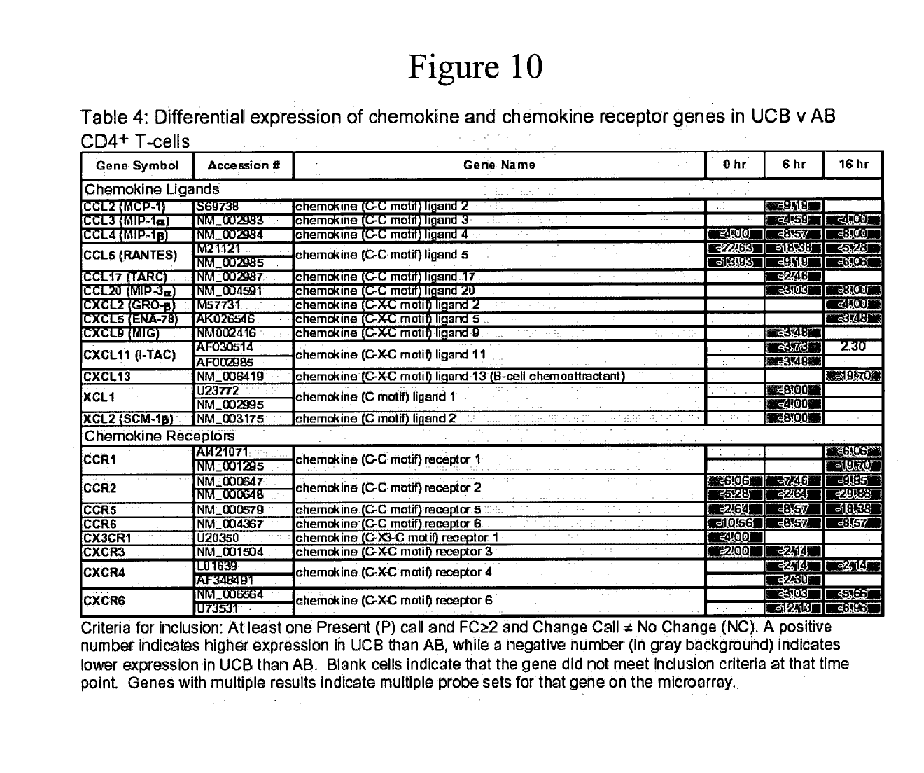

Chemokines and their receptors have been implicated in the pathogenesis of GVHD32 as well as allograft rejection (Fahmy et al. Transplantation. 2003;75:72-78). Additionally, they have been shown to have differential expression in UCB with RANTES, CCR1, CCR2, CCR5, CCR6, and CXCR3 previously described as reduced in UCB (Hariharan et al. Blood. 2000;95:715-718; Sato et al. J Immunol. 2001;166:1659-1666). Applicants therefore queried the array data to determine whether there may be differential expression in additional chemokine and receptor genes between UCB and AB (FIG. 10). Again, with the exception of CXCL11 (IFN-inducible T-cell α chemoattractant (1-TAC)), UCB demonstrated overall lower chemokine mRNA expression than AB. Differences were seen at all time points with most differences seen during stimulation. In absence of stimulation, approximately half of the chemokine receptor genes showed lower expression in UCB with the remaining 18 receptors showing lower expression after stimulation. These data suggest possible impact on the immediate response of UCB T lymphocytes to chemokine signals.

### Example 13

### Confirmation of mRNA Expression

To confirm the data generated from the HG-U133A&B microarrays, RNase protection assays (RPA) were performed analyzing the same RNA pools that were used for the U133AB gene array analysis. Expression patterns of NFAT-dependent genes including IL-2, IL-4, IFN-γ, and p21 were confirmed by RPA (FIG. 13A). Additionally, RPA results for cytokines IL-1β, IL-5, IL-9, IL-10, and receptor IL-1Rβ were noted to corroborate the array data. As shown in FIGS. 13A and 13B, the experimental results of mRNA expression in the RPAs mirror the expression profiles detected by the microarrays (Raw Signal) with, for example, IL-4 showing greatest expression at 6 hr on both the microarray and the RPA, while IL-10 expression continues to increase from 0 to 16 hr.

### Example 14

### Analysis of Protein Expression

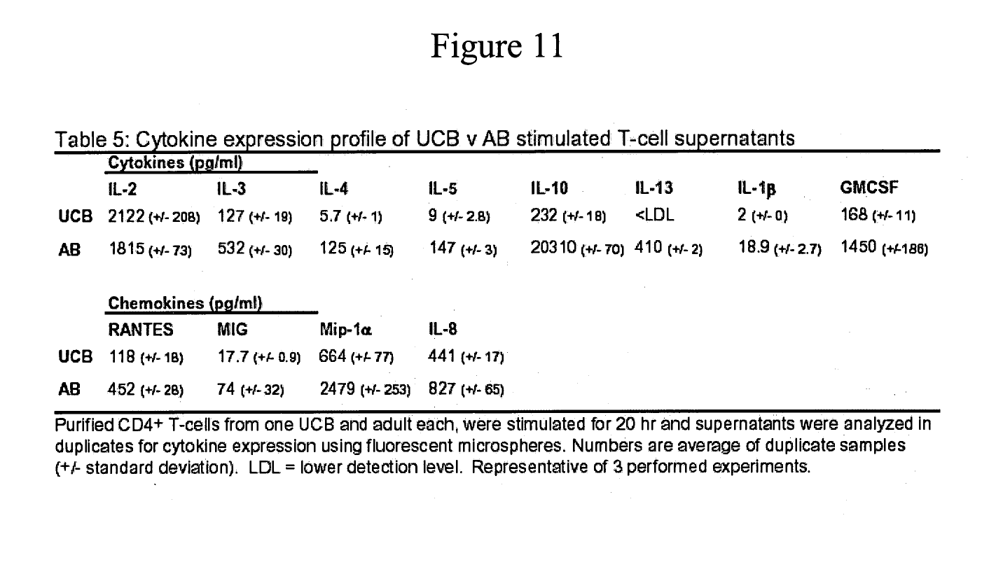

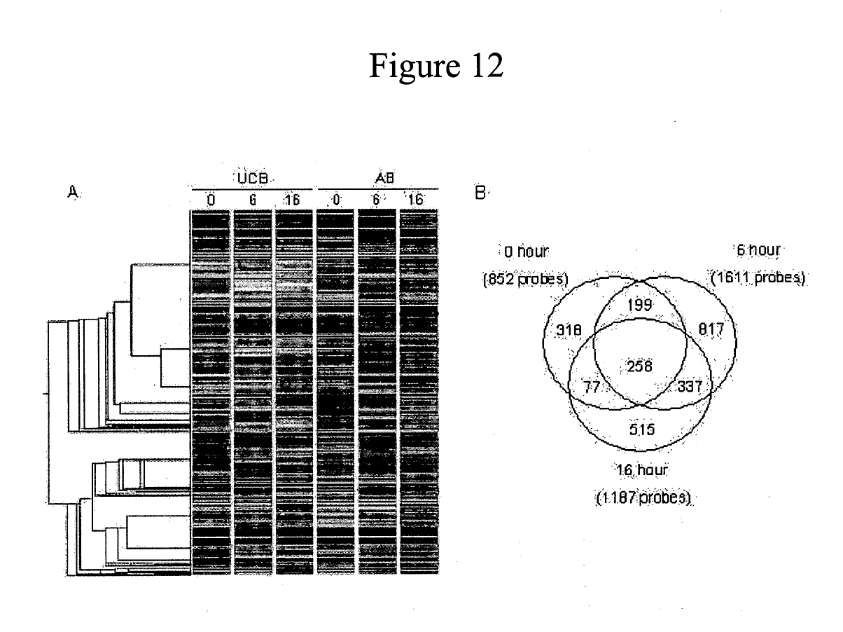

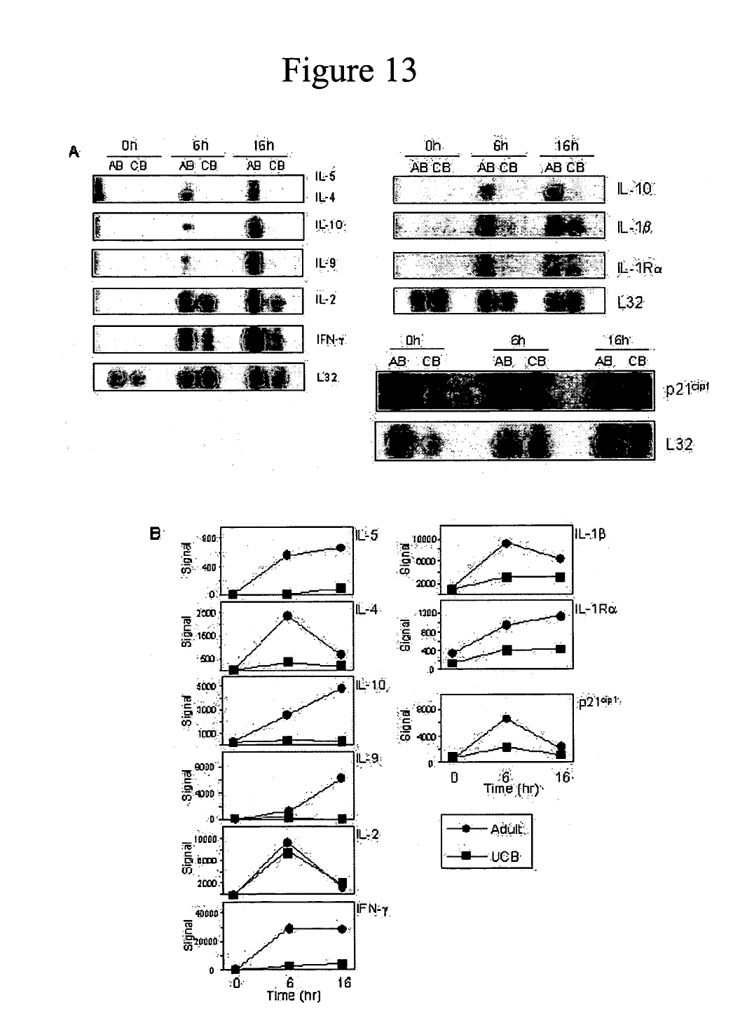

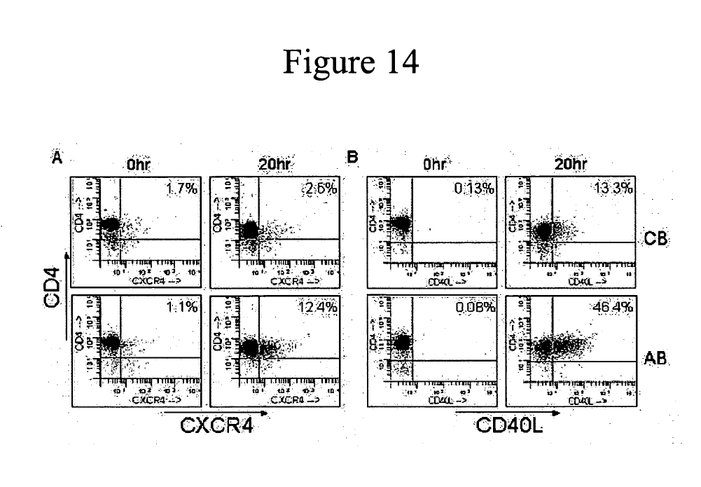

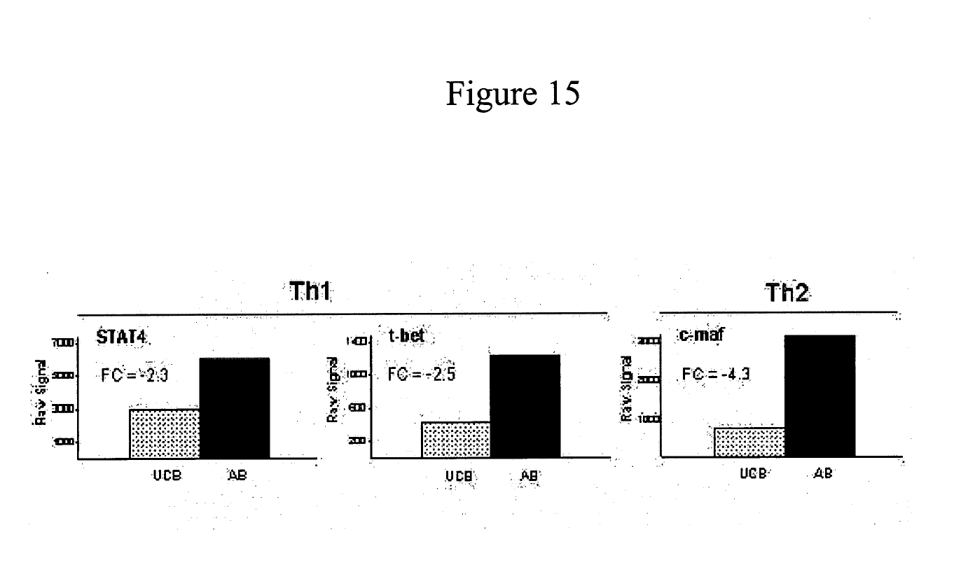

Next, additional confirmatory studies analyzing protein expression by flow cytometry were performed on purified T lymphocytes stimulated for 20 hours. This later time point was chosen to allow for translation of the mRNA into detectable proteins. Correlating with mRNA expression measured by gene array, CXCR4 and CD40L exhibited significantly reduced surface expression in stimulated UCB T lymphocytes. Stimulated cells expressing CXCR4 ranged from 2.1%-3.4% on UCB compared to 2.5%-15.7% on AB ( =4, p<0.05); and expressing CD40L ranged from 0.74%-13.3% on UCB compared to 15%-46.4% on AB (n=4, p<0.05). Representative dot plots are shown in FIG. 14. At 20 hr of stimulation, despite the decreased expression detected on the gene array, CTLA-4 protein surface expression did not differ between UCB and AB. In addition, multiplex cytokine protein measurements were performed using fluorescent microspheres on supernatants obtained from purified T lymphocytes stimulated for 20 hr. NFAT-dependent cytokines including IL-3, IL-4, IL-5, IL-13, GMCSF, and the chemokine MIP-1α demonstrated strongly reduced protein expression in UCB compared to adult supernatant at 20 hr (FIG. 11). In addition to NFAT-dependent genes, IL-1β, IL-10, RANTES, MIG, and IL-8 were found to have strongly reduced protein expression in UCB supernatant. Importantly, IL-2 did not demonstrate differential expression by microarray, RPA or protein analysis, suggesting NFAT independent regulation.

### Example 15

### Impaired Basal Expression of Transcription Factors

With the observed global decrease in both Th1 and Th2 cytokines and chemokines, Applicants explored the gene expression of related transcription factors prior to stimulation. Th1 related transcription factors STAT436 and T-bet37 and Th2-related transcription factor c-maf38,39 demonstrated reduced gene expression in UCB at 0 hr of 2.3-fold, 2.5-fold and 4.3-fold, respectively (FIG. 15). STAT4 and T-bet remained low at 6 hr stimulation in UCB, while c-maf exhibited severely reduced expression at both simulation points. Interestingly, while NFκB mRNA expression was similar at all time points, there was reduced IκBε (NFKBIE) expression in UCB compared to adult at 6 hr stimulation, suggesting potentially reduced NFκB retention in the cytoplasm at later time points of stimulation (FIG. 12).

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

1. A method of decreasing NFATc2 activity in a lymphocyte, comprising exposing the lymphocyte to an antagonist of IFN-γ, thereby decreasing the activity of NFATc2 in the T cell.

2. The method of claim 1, wherein the lymphocyte is a T cell.

3. The method of claim 1, wherein decreasing NFATc2 activity comprises decreasing NFATc2 expression.

4. The method of claim 1, wherein the antagonist of IFN-γ is an anti-IFN-γ antibody.

5. The method of claim 1, wherein the antagonist of IFN-γ comprises an siRNA directed to an mRNA which encodes IFN-γ, IFNgammaR1 or IFNgammaR2.

6. The method of claim 1, wherein the antagonist of IFN-γ is a small molecule drug.

7. The method of claim 2, wherein the T cell is exposed to an antagonist of IFN-γ in the presence of antigen presenting cells.

8. The method of claim 2, wherein the T cell is in a subject.

9. The method of claim 8, wherein the T cell is not endogenous to the subject.

10. The method of claim 9, wherein the T cell, or a progenitor cell thereof, has been transplanted into the subject.

11. The method of claim 2, wherein the T cell is in a transplantable material.

12. A method of increasing NFATc2 activity in a lymphocyte, comprising exposing the lymphocyte to an agonist of IFN-γ, thereby increasing the activity of NFATc2 in the T cell.

13. The method of claim 12, wherein the lymphocyte is a T cell.

14. The method of claim 12, wherein increasing NFATc2 activity comprises increasing NFATc2 expression.

15. The method of claim 12, wherein the agonist is an IFN-γ polypeptide.

16. The method of claim 13, wherein the T cell is exposed to an agonist of IFN-γ in the presence of antigen presenting cells.

17. The method of claim 13, wherein the T cell is in a subject.

18. The method of claim 17, wherein the T cell is not endogenous to the subject.

19. The method of claim 18, wherein the T cell, or a progenitor cell thereof, has been transplanted into the subject.

20. The method of claim 13, wherein the T cell is in a transplantable material.

21. A method of preventing or reducing immune incompatibility in a subject in need thereof, the method comprising administering to the subject a therapeutically effective amount of an IFN-γ antagonist.

22. The method of claim 21, wherein the subject has or is at risk for having graft versus host disease.

23. The method of claim 21, wherein the subject has or is at risk for having graft rejection.

24. The method of claim 21, wherein the subject is the recipient of a transplant.

25. The method of claim 24, wherein the subject is the recipient of a hematopoietic stem cell transplant.

26. The method of claim 24, wherein the subject is the recipient of a solid organ transplant.

27. The method of claim 21, wherein the antagonist of IFN-γ is an anti-IFN-γ antibody.

28. The method of claim 21, wherein the antagonist of IFN-γ comprises a double-stranded RNA.

29. The method of claim 28, wherein the double stranded RNA is an siRNA.

30. The method of claim 29, wherein the siRNA is directed to an mRNA which encodes IFN-γ, IFNgammaR1 or IFNgammaR2.

31. The method of claim 21, wherein the antagonist of IFN-γ is a small molecule drug.

32. The method of claim 24, wherein the transplant is HLA-matched or HLA-unmatched.

33. The method of the claim 24, wherein the transplant is an allogeneic transplant.

34. The method of claim 24, wherein the transplant is lung, heart, kidney, liver, skin, or bone marrow.

35. The method of claim 24, wherein the transplant comprises hematopoetic stem cells from an unrelated donor, umbilical vein hematopoetic stem cells, or peripheral blood stem cells.

36. A method of preventing graft versus host disease in a subject in need of such treatment, the method comprising contacting a transplant, prior to transplantation into the subject, with an IFN-γ antagonist, thereby preventing graft versus host disease in the subject.

37. The method of claim 36, wherein the transplant is HLA-matched or HLA-unmatched.

38. The method of the claim 36, wherein the transplant is an allogeneic transplant.

39. The method of claim 36, wherein the transplant is a solid organ.

40. The method of claim 36, wherein the transplant is lung, heart, kidney, liver, skin, or bone marrow.

41. The method of claim 36, wherein the transplant comprises hematopoietic stem cells.

42. The method of claim 36, wherein the transplant comprises hematopoetic stem cells from an unrelated donor, umbilical vein hematopoetic stem cells, or peripheral blood stem cells.

43. The method of claim 36, wherein the antagonist of IFN-γ is an anti-IFN-γ antibody.

44. The method of claim 36, wherein the antagonist of IFN-γ comprises an siRNA directed to an mRNA which encodes IFN-γ, IFNgammaR1 or IFNgammaR2.

45. The method of claim 36, wherein the antagonist of IFN-γ is a small molecule drug.

46. A method of treating an autoimmune disease in a subject in need thereof, the method comprising administering to the subject a therapeutically effective amount of an IFN-γ antagonist.

47. The method of claim 43, wherein the autoimmune disease is selected from among the following: primary myxoedema, thyrotoxicosis, pernicious anaemia, autoimmune atrophic gastris, Addison's disease, IDDM, Goodpasture's syndrome, myasthenia gravis, sympathetic ophthalmia, MS, autoimmune haemolytic anaemia, idiopathic leucopenia, ulcerative colitis, derinatomyositis, sclerodenna, mixed connective tissue disease, rheumatoid arthritis, irritable bowel syndrome, SLE, Hashimoto's disease, thyroiditis, Behcet's disease, coeliac disease/dermatitis herpetifortnis, and demyelinating disease.

48. The method of claim 46, wherein the antagonist of IFN-γ is an anti-IFN-γ antibody.

49. The method of claim 47, wherein the antagonist of IFN-γ comprises an siRNA directed to an mRNA which encodes IFN-γ, IFNgammaR1 or IFNgammaR2.

50. The method of claim 46, wherein the antagonist of IFN-γ is a small molecule drug.

51. A method of decreasing production of an NFATc2-dependent cytokine in a subject in need of such treatment, comprising administering to the subject a therapeutically effective amount of an IFN-γ antagonist, thereby decreasing the production of the NFATc2-dependent cytokine.

52. A method of increasing production of an NFATc2-dependent cytokine in a T cell in a subject in need of such treatment, comprising administering to the subject a therapeutically effective amount of an IFN-γ agonist polypeptide in the subject, thereby increasing the production of the NFATc2-dependent cytokine in the T cell.

53. The method of claim 51 or 52, wherein the NFATc2-dependent cytokine is selected from the group comprising IFN-γ, TNF-α and IL-2.

54. The method of claim 51, wherein the subject is afflicted with an autoimmune disease.

55. The method of claim 51, wherein the autoimmune disease is selected from the group comprising of primary myxoedema, thyrotoxicosis, pernicious anaemia, autoimmune atrophic gastris, Addison's disease, IDDM, Goodpasture's syndrome, myasthenia gravis, sympathetic ophthalmia, MS, autoimmune haemolytic anaemia, idiopathic leucopenia, ulcerative colitis, derinatomyositis, sclerodenna, mixed connective tissue disease, rheumatoid arthritis, irritable bowel syndrome, SLE, Hashimoto's disease, thyroiditis, Behcet's disease, coeliac disease/dermatitis herpetifortnis, and demyelinating disease.

56. The method of claim 51, wherein the antagonist of IFN-γ is an anti-IFN-γ antibody.

57. The method of claim 51, wherein the antagonist of IFN-γ comprises an siRNA directed to an mRNA which encodes IFN-γ, IFNgammaR1 or IFNgammaR2.

58. The method of claim 51, wherein the antagonist of IFN-γ is a small molecule drug.

59. The method of claim 52, wherein the IFN-γ agonist is an IFN-γ polypeptide.

60. The method of claim 52, wherein the subject is afflicted with a hyperplastic condition or with a viral infection.

61. The method of claim 52, wherein the NFATc2-dependent cytokine is not IFN-γ.

62. A method of preventing or reducing immune incompatibility in a subject in need thereof, the method comprising administering to the subject a therapeutically effective amount of an inhibitor RNA construct that decreases expression of NFATc2 or an NFATc2-regulated factor.

63. The method of claim 62, wherein the subject has or is at risk for having graft versus host disease.

64. The method of claim 62, wherein the subject has or is at risk for having graft rejection.

65. The method of claim 62, wherein the subject is the recipient of a transplant.

66. The method of claim 65, wherein the subject is the recipient of a hematopoietic stem cell transplant.

67. The method of claim 65, wherein the subject is the recipient of a solid organ transplant.

68. The method of claim 65, wherein the transplant is HLA-matched or HLA-unmatched.

69. The method of the claim 65, wherein the transplant is an allogeneic transplant.

70. The method of claim 65, wherein the transplant is lung, heart, kidney, liver, skin, or bone marrow.

71. The method of claim 65, wherein the transplant comprises hematopoetic stem cells from an unrelated donor, umbilical vein hematopoetic stem cells, or peripheral blood stem cells.

72. The method of claim 62, wherein the NFATc2-regulated factor is selected from among the following: IL-3, IL-4, IL-5, IL-13, GM-CSF, IFN-γ, TNF-α, CD40L and MIP-1α.

73. A method of assessing a candidate activator of NFATc2, the method comprising:
a) providing a candidate agent that is an IFN-γ agonist; and
b) measuring an effect of the candidate agent on an NFATc2 activity.

74. The method of claim 73, wherein measuring an effect of the agent on an NFATc2 activity comprises measuring expression of NFATc2 or an NFATc2-regulated gene in an umbilical cord blood T cell culture.

75. The method of claim 73, wherein providing a candidate agent that is an IFN-γ agonist comprises screening a plurality of agents to identify an agent having IFN-γ agonist activity.

76. The method of claim 73, wherein providing a candidate agent that is an IFN-γ agonist comprises obtaining a previously known IFN-γ agonist.

77. The method of claim 73, further comprising, evaluating the effect of the candidate agent on graft versus host disease in an animal.

78. The method of claim 73, further comprising, evaluating the effect of the candidate agent on transplant rejection in an animal.

79. A method of assessing a candidate inhibitor of NFATc2, the method comprising:
a) providing a candidate agent that is an IFN-γ antagonist; and
b) measuring an effect of the candidate agent on an NFATc2 activity.

80. The method of claim 79, wherein measuring an effect of the agent on an NFATc2 activity comprises measuring expression of NFATc2 or an NFATc2-regulated gene in an umbilical cord blood T cell culture.

81. The method of claim 79, wherein providing a candidate agent that is an IFN-γ antagonist comprises screening a plurality of agents to identify an agent having IFN-γ antagonist activity.

82. The method of claim 79, wherein providing a candidate agent that is an IFN-γ antagonist comprises obtaining a previously known IFN-γ antagonist.

83. The method of claim 79, further comprising, evaluating the effect of the candidate agent on graft versus host disease in an animal.

84. The method of claim 83, further comprising, evaluating the effect of the candidate agent on transplant rejection in an animal.
